(19) United States
(12) Patent Application Publication
(10) Pub. No.: US 2025/0281173 A1
(43) Pub. Date: Sep. 11, 2025

(54) KNOTLESS SUTURE ASSEMBLY

(71) Applicant: Arthrex, Inc., Naples, FL (US)

(72) Inventors: Andrew C. Petry, Naples, FL (US); Nirav Patel, Naples, FL (US); Patrick Rudersdorf, Golden, CO (US); Gorav Ailawadi, Ann Arbor, MI (US); Derek C. Sullivan, Naples, FL (US); Jason Valentin, Fort Myers, FL (US)

(73) Assignee: Arthrex, Inc., Naples, FL (US)

(21) Appl. No.: 19/073,343

(22) Filed: Mar. 7, 2025

Related U.S. Application Data
(60) Provisional application No. 63/563,037, filed on Mar. 8, 2024.
(60) Provisional application No. 63/682,911, filed on Aug. 14, 2024.
(60) Provisional application No. 63/753,635, filed on Feb. 4, 2025.

(51) Int. Cl. A61B 17/06; A61B 17/00; A61B 17/04

(52) U.S. Cl. CPC A61B 17/06166; A61B 17/0401; A61B 2017/00526; A61B 2017/0406

(57) ABSTRACT

A knotless suture assembly may include a repair suture which may have a first and a second end. The first end may be coupled to a first needle. The knotless suture assembly may also include a shuttle suture. The shuttle suture may have a first end coupled to the first needle and a second end coupled to a second needle. The shuttle suture may include a shuttling feature, and the first end of the repair suture may be configured to engage the shuttling feature during a shuttling process. The knotless suture assembly may include a locking feature. The repair suture may be configured to engage the locking feature during the shuttling process. The locking feature may be configured to retain the repair suture under tension in response to the shuttling process.

### CROSS-REFERENCE TO RELATED APPLICATIONS

This application claims priority under 35 U.S.C. § 119 (e) and the benefit of U.S. Provisional Application No. 63/563,037 entitled KNOTLESS VALVE SUTURE, filed on Mar. 8, 2024, U.S. Provisional Application No. 63/682,911 entitled KNOTLESS VALVE SUTURE, filed on Aug. 14, 2024, and U.S. Provisional Application No. 63/753,635 entitled KNOTLESS SUTURE ASSEMBLY WITH PREFORMED SPLICED SEGMENTS, filed on Feb. 4, 2025, the entire disclosures of which are incorporated herein by reference.

### BACKGROUND

The present disclosure generally relates to a suture assembly and, more particularly, to a knotless suture assembly including a repair suture and shuttle suture for cardiovascular valve procedures. During cardiovascular procedures, such as valve repairs or valve replacements, sutures can be used to attach a valve to tissue. The sutures are often knotted to hold the suture under tension. In other aspects, a crimp can be placed over the suture and crimped or deformed to hold the suture under tension. The sutures can form loops or stitches through a shuttling process. In various implementations, the disclosure provides for devices and methods that may assist in the operation of suture assemblies by improving the locking structure and/or improving how the suture is carried through tissue.

### SUMMARY

In various aspects, knotless suture assemblies may be utilized to connect valves to tissue, connect multiple tissue bodies, and close cuts or openings in tissue. Knotless suture assemblies may include a repair suture, a shuttle suture, and one or more needles coupled to the repair suture and/or the shuttle suture. The needle(s) may be used to carry the sutures through tissue and/or a replacement valve concurrently. The sutures may be coupled with a pledget, which can be positioned against an underside of the tissue. The shuttle suture may be used for carrying the repair suture through a locking feature for providing a knotless stitch configuration.

According to an aspect of the present disclosure, a knotless valve suture assembly may include a pledget. A repair suture may have a proximal end fixedly coupled to the pledget and a distal end, which may be coupled to a first needle. A shuttle suture may extend through the pledget. The shuttle suture may be coupled with the first needle and a second needle. The shuttle suture may include a shuttling feature proximate to the second needle. A locking feature may be coupled to the pledget. The shuttling feature may be a shuttle link, a receiving area of the shuttle suture, a shuttle loop, and/or an area formed by a folded shuttle suture. The locking feature may be configured to retain the repair suture under tension when the repair suture is threaded through the locking feature in response to a shuttling process. The locking feature may be a spliced segment of the repair suture and/or a grommet. In certain aspects, the pledget may define a ring shape and multiple suture pairs, which each may include a repair suture and a shuttle suture, may be coupled to the pledget with the ring shape.

In certain aspects, the knotless suture assembly may include a first end of the shuttle suture may form the shuttling feature. The shuttle suture may be spliced into the repair suture at a first spliced segment proximate a first end of the repair suture and at a second spliced segment proximate to a second end of the repair suture. A stuffing suture may be spliced into the repair suture between the first and second spliced segments between the repair suture and the shuttle suture. The first and second spliced segments may be spaced from first and second ends of the shuttle suture and/or may include the first and second ends of the shuttle suture. One or both ends of the suture assembly may be coupled with a respective needle for insertion through tissue. The shuttle suture may be configured to guide the repair suture through the first spliced segment which can, consequently, bunch the stuffing suture to lock the repair suture in a stitch configuration.

In additional aspects, a method of manufacturing the knotless suture assembly including the spliced segments may include cutting suture material to a predefined length. A repair suture may be provided or formed. A shuttle suture may be provided and spliced into the repair suture. The shuttle suture may be spliced into the repair suture at one or more locations, with at least one spliced segment forming a locking feature for retaining the repair suture under tension in response to a shuttling process. Often, a lead end of the shuttle suture may be spliced into the repair suture to form at least one combined or spliced end of the suture assembly for inserting the suture assembly through tissue.

In various aspects, the knotless suture assembly may be utilized in various cardiovascular and orthopedic procedures. For example, the knotless suture assembly may be utilized in a method of replacing a cardiovascular valve. Additionally or alternatively, the knotless suture assembly may be used in a method of compressing tissue bodies to reduce a gap or opening therebetween.

These and other features, objects, and advantages of the present disclosure will become apparent upon reading the following description thereof together with reference to the accompanying drawings.

### BRIEF DESCRIPTION OF THE DRAWINGS

FIG. 1 is representative of a cardiovascular valve replacement with sutures to bring a replacement valve to engage an annulus, according to the present disclosure;

FIG. 2 is representative of an aortic annulus with suture assemblies arranged around the annulus, according to the present disclosure;

FIG. 3 is a schematic diagram of a suture assembly extending through tissue and a replacement valve, where the suture assembly includes a pledget under the tissue, a repair suture, a shuttle suture, and two needles, according to the present disclosure;

FIG. 4 is a schematic diagram of a suture assembly with a repair suture being threaded through a shuttle link of a shuttle suture, according to the present disclosure;

FIG. 5 is a schematic diagram of a suture assembly with a repair suture threaded through a shuttle link of a shuttle suture, illustrating a pulling force being applied to the shuttle suture to guide the repair suture, according to the present disclosure;

FIG. 6 is a schematic diagram of a suture assembly with a repair suture threaded through a spliced section of the repair suture to lock the repair suture, according to the present disclosure;

FIG. 7 is a schematic diagram of a suture assembly extending through tissue and a replacement valve, where the suture assembly includes a folded pledget under the tissue, a repair suture, a shuttle suture, and two needles, where the folded pledget is illustrated in an expanded state, according to the present disclosure;

FIG. 8 is a schematic diagram of a suture assembly extending through tissue and a replacement valve, where the suture assembly includes a folded pledget under the tissue, a repair suture, a shuttle suture, and two needles, where the folded pledget is illustrated in a compressed state, according to the present disclosure;

FIG. 9 is a schematic diagram of a suture assembly including an annular pledget and multiple suture pairs with a repair suture and a shuttle suture, according to the present disclosure;

FIG. 10 is a schematic diagram of a suture assembly with a repair suture threaded through a shuttle link of a shuttle suture, illustrating a pulling force being applied to the shuttle suture to guide the repair suture through a grommet for locking the repair suture, according to the present disclosure;

FIG. 11 is a flow diagram of a method of using a knotless suture assembly with a shuttle link in a valve replacement procedure, according to the present disclosure;

FIG. 12 is a schematic diagram of a suture assembly extending through tissue and a replacement valve, where the suture assembly includes a folded pledget under the tissue, a repair suture, a shuttle suture, and two needles, where the shuttle suture includes a shuttle receiving area;

FIG. 13 is a schematic diagram of a suture assembly extending through tissue and a replacement valve, where the suture assembly includes a folded pledget under the tissue, a repair suture, a shuttle suture, and two needles, where the repair suture is inserted through the shuttle suture at a shuttle receiving area;

FIG. 14 is a flow diagram of a method of using a knotless suture assembly with a shuttle receiving area in a valve replacement procedure;

FIG. 15 is a schematic diagram of a suture assembly extending through tissue and a replacement valve, where the suture assembly includes a folded pledget under the tissue, a repair suture, a shuttle suture, and two needles, where the shuttle suture is folded to form a shuttle loop, according to the present disclosure;

FIG. 16 is a schematic diagram of a suture assembly extending through tissue and a replacement valve, where the suture assembly includes a folded pledget under the tissue, a repair suture, a shuttle suture, and two needles, where the repair suture is inserted through a shuttle loop of the shuttle suture, according to the present disclosure;

FIG. 17 is a flow diagram of a method of using a knotless suture assembly with a shuttle loop in a valve replacement procedure, according to the present disclosure;

FIG. 18 is a schematic diagram of a suture assembly with needles coupled to ends of a repair suture and ends and a mid-region of a shuttle suture spliced into the repair suture, according to the present disclosure;

FIG. 19 is a schematic diagram of the suture assembly of FIG. 18 with the needles removed and the ends of the repair suture and the shuttle suture separated, according to the present disclosure;

FIG. 20 is a schematic diagram of a suture assembly with needles coupled to ends of a repair suture, ends and a mid-region of a shuttle suture spliced into the repair suture, and a pledget, according to the present disclosure;

FIG. 21 is a schematic diagram of a repair suture for a suture assembly, according to the present disclosure;

FIG. 22 is a schematic diagram of a repair suture and a shuttle suture forming a suture assembly, according to the present disclosure;

FIG. 23 is a schematic diagram of a suture assembly with a shuttle suture and a stuffing suture spliced into a repair suture, according to the present disclosure;

FIG. 24 is a method of manufacturing a suture assembly, according to the present disclosure;

FIG. 25 is a schematic diagram of a suture assembly being inserted through a tissue body, according to the present disclosure;

FIG. 26 is a schematic diagram of a suture assembly being inserted through adjacent tissue bodies, according to the present disclosure;

FIG. 27 is a schematic diagram of a suture assembly inserted through adjacent tissue bodies with a spliced region between a repair suture and a shuttle suture arranged below the tissue bodies, according to the present disclosure;

FIG. 28 is a schematic diagram of a suture assembly inserted through adjacent tissue bodies with a repair suture engaging a shuttle loop of a shuttle suture, according to the present disclosure;

FIG. 29 is a schematic diagram of a suture assembly with a repair suture in a stitch configuration from a shuttling process, according to the present disclosure;

FIG. 30 is a schematic diagram of a repair suture providing compression between adjacent tissue bodies, according to the present disclosure;

FIG. 31 is a schematic diagram of a repair suture tensioned to compress adjacent tissue bodies together, according to the present disclosure;

FIG. 32 is a schematic diagram of a suture assembly being inserted through a tissue body, according to the present disclosure;

FIG. 33 is a schematic diagram of a suture assembly being inserted through overlapping tissue bodies, according to the present disclosure;

FIG. 34 is a schematic diagram of a suture assembly inserted through overlapping tissue bodies with a spliced region between a repair suture and a shuttle suture arranged below an inner layer of the overlapping tissue bodies, according to the present disclosure;

FIG. 35 is a schematic diagram of a suture assembly inserted through overlapping tissue bodies with a repair suture engaging a shuttle loop of a shuttle suture, according to the present disclosure;

FIG. 36 is a schematic diagram of a suture assembly with a repair suture in a stitch configuration from a shuttling process, according to the present disclosure;

FIG. 37 is a schematic diagram of a repair suture tensioned to compress overlapping tissue bodies together, according to the present disclosure; and

FIG. 38 is a method of compressing tissue bodies together, according to the present disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

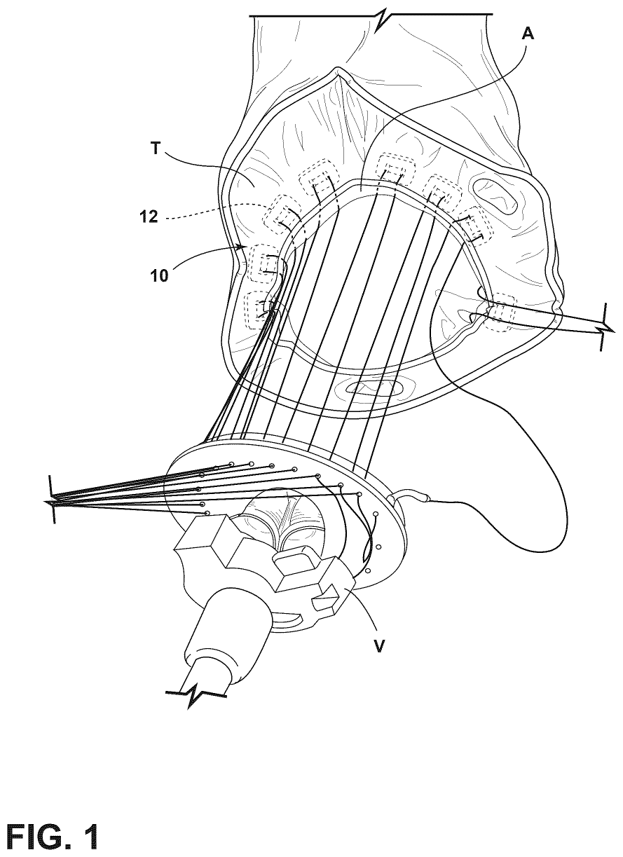

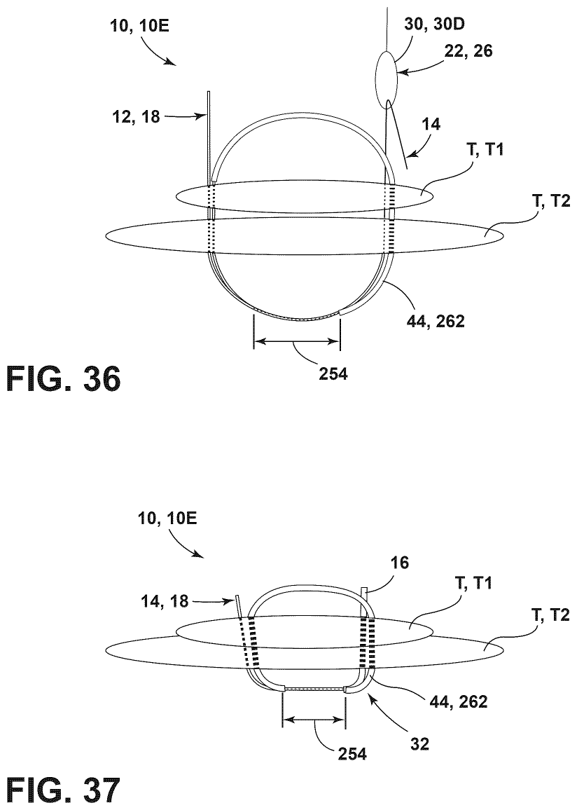

With reference to FIGS. 1-38, reference numeral 10 generally designates a knotless suture assembly, exemplary configurations of which are referred to herein as knotless suture assemblies 10A-10E that can provide configurations for different shuttling approaches and/or approaches for insertion through tissue T and/or implant. The knotless suture assembly 10 may include a pledget 12, which may have various configurations, examples of which are referred to herein as pledgets 12A-12C. The suture assembly 10 may further include a repair suture 14. The repair suture 14 may include a first end 16 and a second end 18. The repair suture 14 may be coupled with at least a first needle 20. A shuttle suture 22 may include a first end 24 and a second end 26 and may extend through the pledget 12. The shuttle suture 22 may be coupled with at least the first needle 20 or both the first needle 20 and a second needle 28.

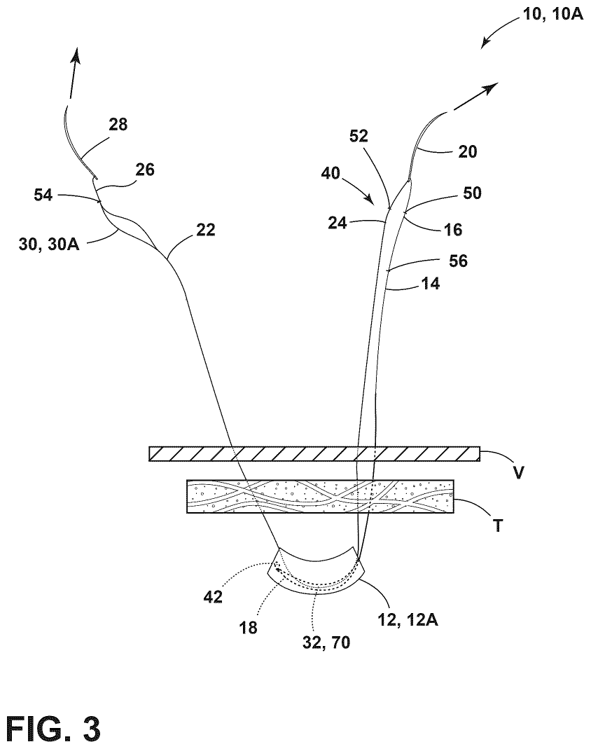

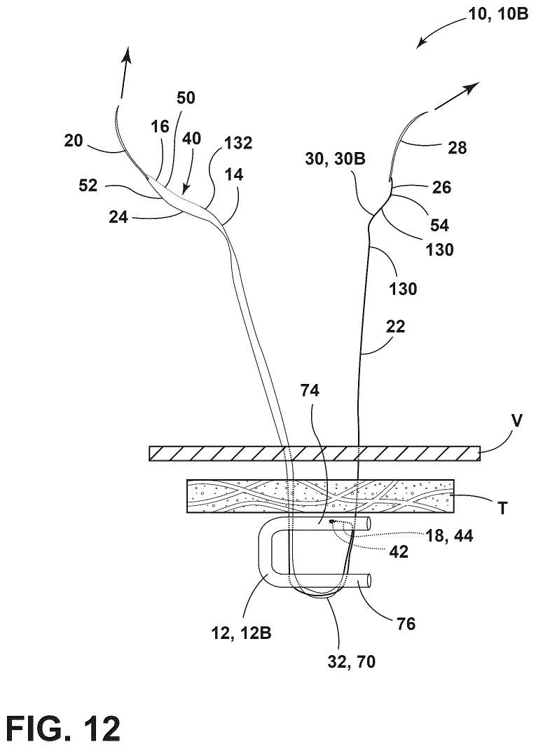

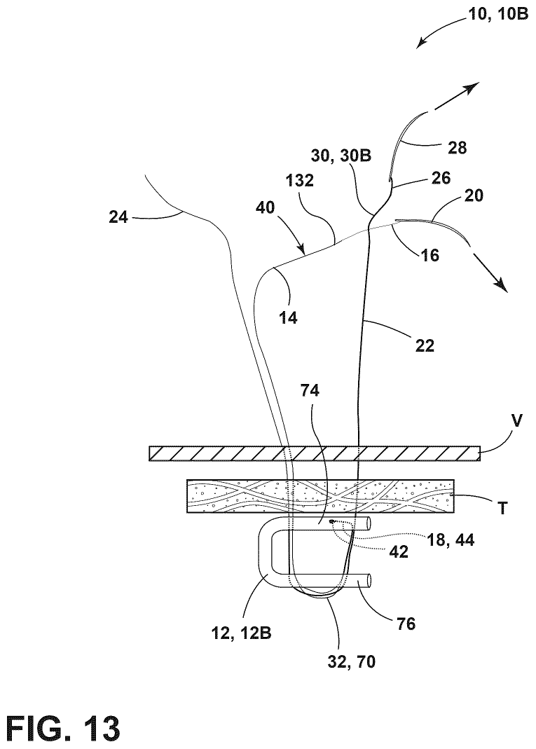

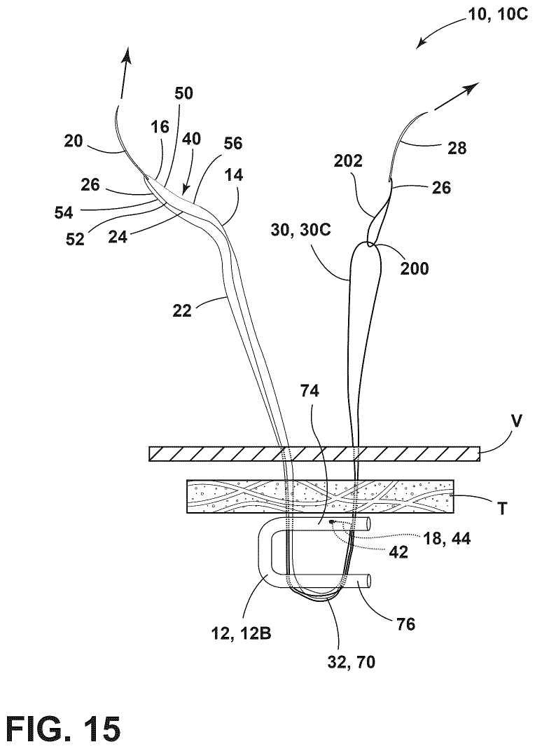

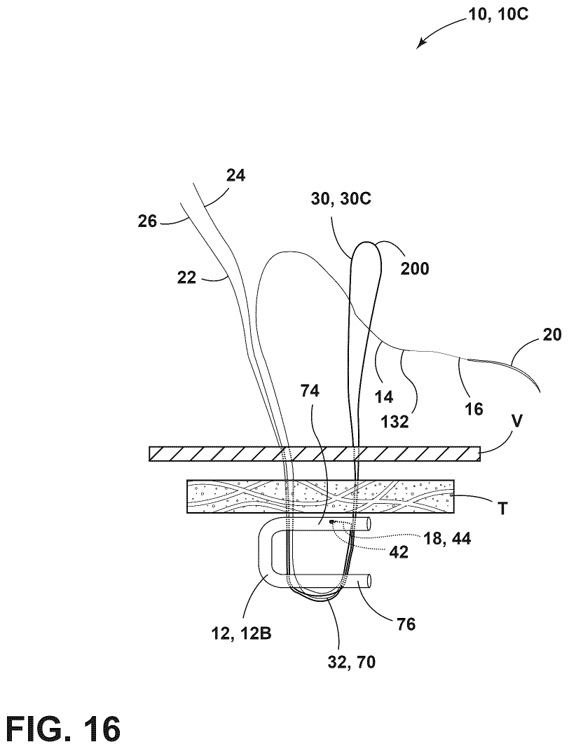

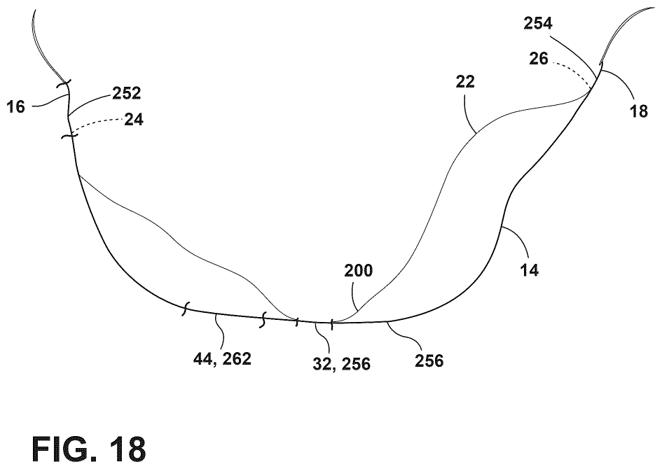

In certain aspects, the shuttle suture 22 and the repair suture 14 may each be connected to the first needle 20 at a common end of the suture assembly 10. As illustrated in various examples, the shuttle suture 22 may also form or include a loop, opening, link, or other shuttling feature(s) 30, which may be proximate to the second needle 28. Examples of the shuttling feature 30 may be referred to herein as a shuttle link 30A (see FIGS. 3-8), a shuttle receiving area 30B (see FIGS. 12 and 13), a folded shuttle loop 30C (see FIGS. 15 and 16), and an end shuttle loop 30D (see FIGS. 18, 19, 21, and 22). Additionally, the knotless suture assembly 10 may include a locking feature 32. The locking feature 32 may be within or coupled to the pledget 12 and/or part of the repair suture 14 to retain the repair suture 14 under tension.

The suture assembly 10 may generally configured for use with connecting devices or implants to muscle/soft tissue T, as well as for connecting tissue T bodies together. In a non-limiting example, the suture assembly 10 may be utilized for heart valve replacements or heart valve repairs, where the implant may be a replacement valve V. In such examples, the suture assembly 10 may be utilized for a mitral valve replacement or an aortic valve replacement. Using the aortic valve replacement as an example, an annulus A extends around leaflets of the aortic valve. In the valve replacement, the leaflets are removed from a patient. The suture assembly 10 may be configured to draw the replacement valve V into the patient to implant and replace the valve that was removed from the patient. Depending on the type of valve replacement, the valve replacement procedure may access the aortic valve via the aorta or the mitral valve via the septum.

The replacement valve V may be a mechanical valve or may be a tissue-based valve, which can often be a xenograft-type of tissue. The type of valve V may be chosen based on various factors, including patient age, patient diagnosis, prior health complications, medications, etc. The suture assembly 10 may be used with any type of replacement valve V without departing from the teachings herein. The suture assembly 10 may assist with defining a fluid-tight or blood-tight seal between the tissue T and the replacement valve V. Moreover, the suture assembly 10 may be re-tensioned, as described herein, during the valve replacement procedure to maximize alignment of the replacement valve V.

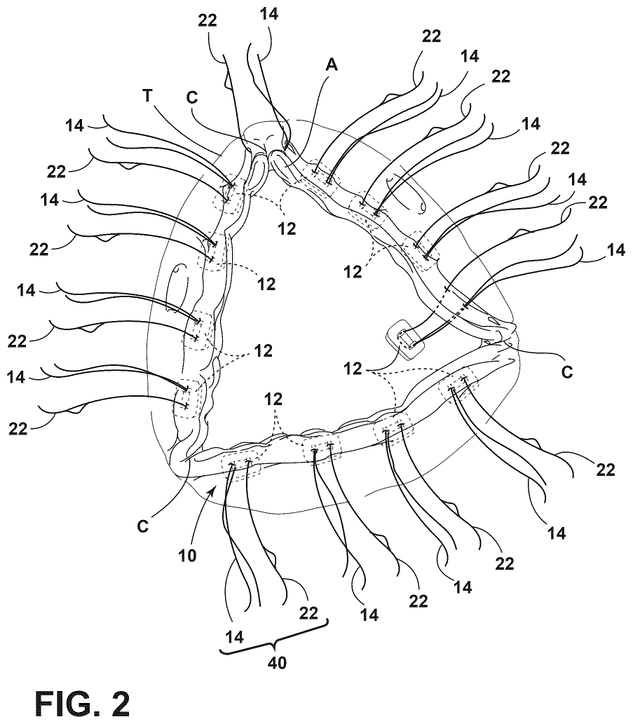

Referring to FIGS. 1 and 2, during the valve replacement procedure, the replacement valve V can be brought to engage and compress against the tissue T. The replacement valve V may generally be sized to correspond with a size of the annulus A or a tube that the replacement valve V is being sewn into. For example, if the opening for the tube has a 27 mm diameter, a 27 mm replacement valve V may be used to reduce or prevent any gaps outside of the replacement valve V.

Multiple suture assemblies 10, including any one or more of the exemplary configurations disclosed herein, may be used to secure the replacement valve V to the tissue T. For example, between about 12 and about 17 suture pairs 40, each generally including one repair suture 14 and one shuttle suture 22, may be utilized. The suture pairs 40 can be spaced around the annulus A to secure the replacement valve V to the tissue T. The knotless aspect of the suture assembly 10 may be advantageous for more efficiently securing the replacement valve V to the tissue T while reducing components arranged on the replacement valve V, where conventional knots and crimps can be positioned.

Referring to FIG. 3, the suture assembly 10 may include the repair suture 14 and the shuttle suture 22. The repair suture 14 may be used to attach the replacement valve V to the tissue T, while the shuttle suture 22 may be utilized to shuttle or guide the repair suture 14 to form a stitch upon the application of a pulling force or tension. The configuration of the shuttle suture 22 and/or the arrangement of the shuttle suture 22 relative to the pledget 12 may provide different shuttling approaches. The suture assembly 10 may also include the pledget 12, and each of the repair suture 14 and the shuttle suture 22 may be operably coupled with or engaged with the pledget 12.

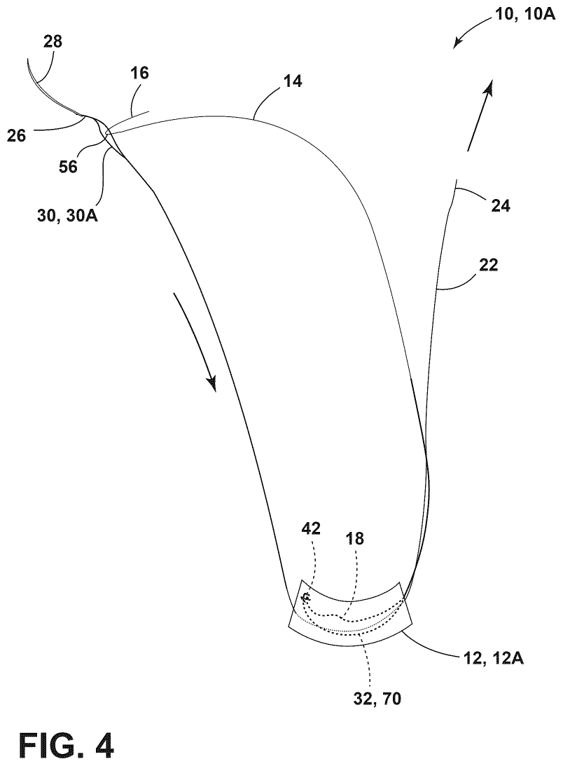

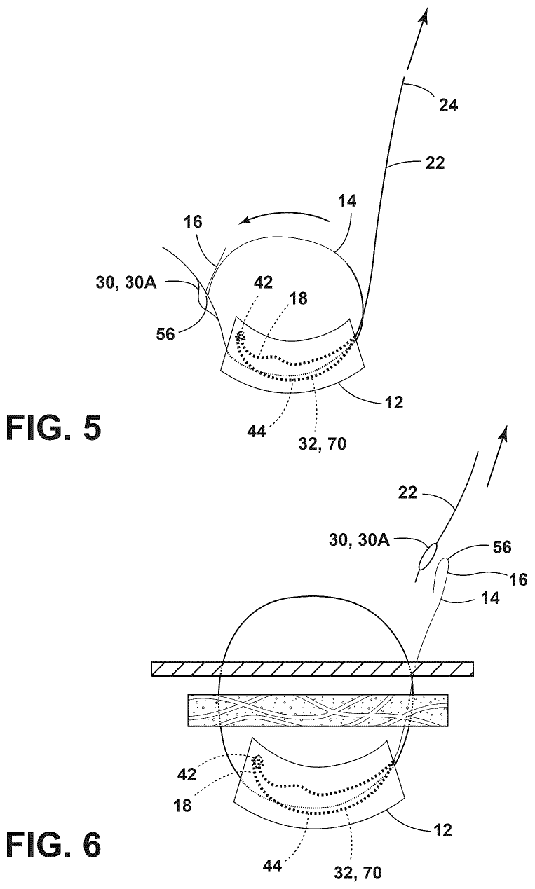

The first end 16 of the repair suture 14 may be coupled with the first needle 20. In this configuration, the first end 16 may also be referred to as a distal end, a tail end, or a tail. The distal end 16 may be spaced from the pledget 12 to provide a length for the repair suture 14 to form the stitch, be tensioned, and be re-tensioned. In various aspects, the repair suture 14 may have a greater thickness or width at the proximal end 18 and a tapered width at the distal end 16. The proximal end 18 may include stuffing 44 or stuffing suture 44 (see FIG. 5), which may be additional suture material stuffed within the repair suture 14 to increase the width or thickness to assist with locking the repair suture 14. The distal end 16 may be tapered through the manufacturing process. For example, in aspects where the repair suture 14 is woven together, fewer strands of material may be used to form the distal end 16. The tapered distal end 16 may assist with the distal end 16 being carried by the shuttle suture 22 during the shuttling process as described later herein with reference to FIGS. 4-6.

In various aspects, the repair suture 14 may include the second end 18, which may be referred to as a proximal end, a base, or a base end. The proximal end 18 may be fixed to the pledget 12 via a base knot 42. The base knot 42 may be positioned proximate to a first side of the pledget 12, and the repair suture 14 may extend along the pledget 12 to exit the pledget 12 proximate to an opposing second side.

Referring still to FIG. 3, for the illustrated knotless suture assembly 10A, the shuttle suture 22 may include the first end 24, which may be referred to as a tensioning end, and the second end 26, which may be referred to as a guiding or shuttling end. The ends 24, 26 of the shuttle suture 22 may both be spaced from the pledget 12, while a central or middle section of the shuttle suture 22 may extend through the pledget 12. The first end 24 of the shuttle suture 22 may be coupled with the first needle 20. In this way, both the distal end 16 of the repair suture 14 and the first end 24 of the shuttle suture 22 may be coupled to the first needle 20. The first end 24 of the shuttle suture 22 and the distal end 16 of the repair suture 14 may both be coupled to the first needle 20 such that there are two tail ends coupled with the needle 20. Alternatively, the first end 24 of the shuttle suture 22 may be spliced into the repair suture 14 for a single tail (e.g., the distal end 16 or the combined ends 16, 24) to be coupled to the needle 20, similar to as described later herein with respect to FIGS. 18-20 and FIGS. 22-24.

Additionally, the second end 26 of the shuttle suture 22 may be coupled with the second needle 28, such that both ends 24, 26 of the shuttle suture 22 may be coupled with needles 20, 28, respectively. The second end 26 of the shuttle suture 22 may define or include the shuttle link 30A, which may be an opening or loop through which the repair suture 14 can be inserted and wrapped to pass through the locking feature 32 as later described in reference to FIGS. 4-6. In non-limiting examples, the shuttle link 30A may be formed by a bifurcated segment of the shuttle suture 22 at the second end 26. In such examples, during the manufacturing process, the shuttle suture 22 may be woven, braised, interlaced, or otherwise formed as a single limb, then formed as two limbs to form the bifurcated segment, and then again formed as the single limb to form the shuttle link 30A. This process may be advantageous for retaining a smaller width of the shuttle suture 22 at the shuttle link 30A. With the bifurcated segment forming the shuttle link 30A, the width of the bifurcated segment may be substantially similar to the remainder of the shuttle suture 22. Additionally, the shuttle suture 22 may have a single tail adjacent to the shuttle link 30A for engaging the needle 28.

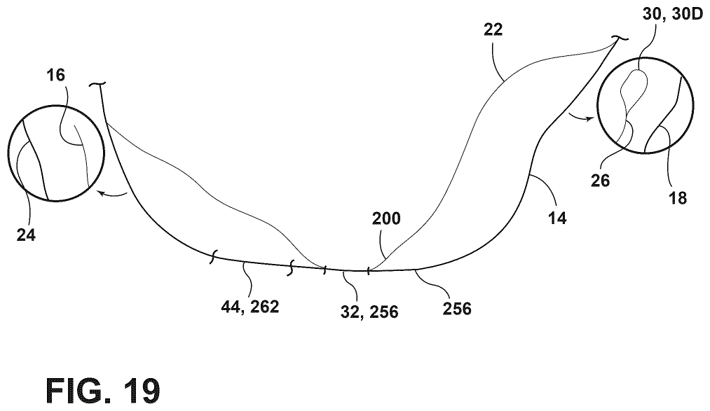

In additional or alternative examples, the shuttle link 30A may be formed as a loop (see, e.g., FIGS. 19, 22, and 23). In such examples, the second end 26 of the shuttle suture 22 may be folded to engage itself to form the loop. In this configuration, the width of the shuttle suture 22 at the loop may be greater than the width of the remainder of the shuttle suture 22. Alternatively, the shuttle suture 22 may be manufactured to taper at the second end 26 to reduce the size of the loop. For example, the tapered end 26 forming the loop may have a width or thickness substantially equal to the remainder of the shuttle suture 22.

Referring still to FIG. 3, one or both of the repair suture 14 and the shuttle suture 22 may have cut markers or indicators 50-54 proximate to the needles 20, 28. The repair suture 14 may have the cut indicator 50 at the distal end 16 proximate to the first needle 20. The shuttle suture 22 may have cut indicators 52, 54 at each of the first end 24 and the second end 26, respectively. At the second end 26, the cut indicator 54 may be between the shuttle link 30A and the second needle 28.

The cut indicators 50-54 may provide a location for separating the needles 20, 28 from the sutures 14, 22 after the suture assembly 10 is engaged with the tissue T and/or the implant/replacement valve V. The cut indicators 50-54 may generally leave a similar length of the repair suture 14 and two lengths of the shuttle suture 22 extending from the replacement valve V to be used to form the stitch. Upon removal of the needles 20, 28, the distal end 16 of the repair suture 14 and the first end 24 of the shuttle suture 22 may be separated from one another. Further, depending on the configuration of the shuttle link 30A, upon removal of the second needle 28, the second end 26 of the shuttle suture 22 may end in a tail section extending from the bifurcated segment or may end in the loop.

Referring still to FIG. 3, the pledget 12 may generally be a mesh-like or braided component that can assist with distributing pressure from the sutures 14, 22 along a greater surface area of the tissue T. The pledget 12 may form a buttress for the suture assembly 10 against the tissue T. In this configuration, the repair suture 14 may be fixed to the pledget 12 and may extend at least partially through the pledget 12. The shuttle suture 22 may extend through the pledget 12 and can be removed from the pledget 12 during the shuttling process. The braided or mesh configuration of the pledget 12 may allow the sutures 14, 22 to be passed through the pledget 12 to form the stitch.

Referring now to FIGS. 3 and 4, for example, when used for a replacement valve procedure, the pledget 12 may be arranged along an inner surface of the tissue T or under the annulus A. The suture assembly 10A may include two needles 20, 28 provided to pass the ends 16, 18, 24, 26 of the sutures 14, 22 through the tissue T from a first side to an opposing second side and through the replacement valve V. The first needle 20 can be inserted through the tissue T from the underside of the annulus A and then through the replacement valve V, which may bring the distal end 16 of the repair suture 14 and the first end 24 of the shuttle suture 22 through the tissue T and the replacement valve V. Similarly, the second needle 28 can be inserted through the tissue T from the underside of the annulus A and then through the replacement valve V, which may bring the second end 26 of the shuttle suture 22 through the tissue T and the replacement valve V. The sutures 14, 22 extending through the tissue T may be spaced by approximately the length of the pledget 12.

The use of two needles 20, 28 may allow the suture assembly 10 to be packaged in a preassembled configuration ready for application in a sterile setting. The two needles 20, 28 and corresponding engagement to the sutures 14, 22 may allow the pledget 12 to remain under the annulus A to distribute pressure caused by the sutures 14, 22. Further, the use of two needles 20, 28 can assist with strengthening an engagement between the tissue T and the replacement valve V to provide a better seal and compression of the replacement valve V into the tissue T. For example, with the aortic valve replacement, there is generally a high gradient of blood flow that moves through the aortic valve into the body of the patient to be able to pump blood through the extremities of the patient. The two pass-throughs of the suture assembly 10 through the tissue T and the replacement valve V may assist in providing a blood-tight seal between the tissue T and the replacement valve V with greater compression of the replacement valve V against the tissue T.

Referring to FIGS. 4-6, once the needles 20, 28 have been removed, the sutures 14, 22 may be utilized to form the stitch typically via the shuttling process. The distal end 16 of the repair suture 14 may be threaded through the shuttle link 30A. The repair suture 14 may include one or more fold indicators 56, which may be visually differentiated from the cut indicators 50-54 to guide a user in where to cut and where to fold the repair suture 14. The distal end 16 of the repair suture 14 may be inserted through the shuttle link 30A and folded at the fold indicator 56.

The repair suture 14 may generally be folded back to abut and extend along itself to form the folded distal end 16 around the shuttle link 30A. This folded arrangement around the shuttle link 30A may allow the repair suture 14 to remain engaged with the shuttle link 30A as the pulling force is applied to the shuttle suture 22. The distal end 16 of the repair suture 14 being narrower or tapered may be advantageous for forming a smaller folded distal end 16. The smaller folded distal end 16 may be advantageous for guiding the folded distal end 16 through the pledget 12 and/or the locking feature 32 while reducing resistance.

Still referring to FIGS. 4-6, the pulling force can be applied to the first tensioning end 24 of the shuttle suture 22, which may draw the shuttle link 30A and the engaged folded distal end 16 of the repair suture 14 toward and through the replacement valve V and the tissue T. As the proximal end 18 of the repair suture 14 can be coupled to the pledget 12 at the base knot 42, the proximal end 18 may remain in a generally fixed location as the repair suture 14 is looped over the replacement valve V and then guided through the tissue T and the replacement valve V. At this stage, the repair suture 14 may extend through the tissue T and the replacement valve V at two different locations and can extend across the replacement valve V between the two locations. Though discussed primarily in reference to the replacement valve V, it shall be understood that knotless suture assembly 10 may be applied in a variety of applications and procedures.

As in the configuration illustrated in FIG. 5, the suture assembly 10A may include the locking feature 32, which may be configured as a spliced section 70 of the repair suture 14 proximate to the base knot 42. Generally, the spliced section 70 may be disposed within the pledget 12. At the spliced section 70, the shuttle suture 22 may be woven, interlaced, or otherwise extended through the repair suture 14. The repair suture 14 may include the stuffing 44 at or proximate to the spliced section 70. In this configuration, the tension applied to the repair suture through the increased width of the stuffing 44 or padding may cause the stuffing 44 to compress or wrinkle to assist with binding or locking the repair suture 14. In the instant example, the spliced section 70 may be between about five millimeters and about six millimeters in length along the repair suture 14. However, the proportions of the spliced section 70 and various features of the suture assembly 10 may vary depending on the application.

With the tension or pulling force being applied to the first end 24 of the shuttle suture 22, the shuttle suture 22 may be drawn through the spliced section 70. As the shuttle suture 22 is drawn through the spliced section 70, the folded distal end 16 of the repair suture 14 may also be drawn through the spliced section 70. Accordingly, the repair suture 14 may be pulled through itself at the spliced section 70. The tapered distal end 16 may be advantageous for pulling the repair suture 14 through itself with less resistance. The pulling force may cause the shuttle suture 22 to be pulled entirely through the spliced section 70 and may pull the repair suture 14 through the spliced section 70 until the repair suture 14 is tightened or tensioned.

In certain aspects, the padding or stuffing 44 may be added at the base of the repair suture 14 proximate to the base knot 42 and the spliced section 70 to assist with locking the repair suture 14 as the repair suture 14 extends through itself at the spliced section 70. As the repair suture 14 is pulled through itself, the thicker part of the repair suture 14 can bunch or catch to create a locking trap. This locking feature 32 and the resistance caused by the thicker, stuffed portion of the repair suture 14 may lock the repair suture 14 as the repair suture 14 is tensioned.

As illustrated in FIGS. 5 and 6, the pulling force can continue to be applied to the shuttle suture 22 to draw the shuttle suture 22 and the folded distal end 16 of the repair suture 14 through the pledget 12. As shown in FIG. 5, the shuttle suture 22 and the folded distal end 16 of the repair suture 14 can be drawn across the pledget 12, through the tissue T, and through the replacement valve V by the pulling force on the first end 24 of the shuttle suture 22. As shown in FIG. 6, the shuttle suture 22 may be pulled fully from the tissue T and the replacement valve V. The withdrawal of the shuttle suture 22 may further cause the shuttle link 30A to draw the distal end 16 of the repair suture 14 along the same path, such that the distal end 16 is exposed from the tissue. Accordingly, the entire path of the closure loop formed by the repair suture 14 may be as follows: the repair suture 14 may extend from the base knot 42, through the pledget 12 under the annulus A, through the tissue T and the replacement valve V, over the replacement valve V, back through the replacement valve V and the tissue T, through the spliced section 70, and again through the tissue T and the replacement valve V to form the stitch. The resulting exposed distal end 16 of the repair suture 14 may be pulled to tension the suture 14, and the locking feature 32 may automatically lock the suture 14 as the repair suture 14 is tensioned.

The shuttle suture 22 and the repair suture 14 may be pulled through the replacement valve V and the tissue T in the locations where the sutures 14, 22 are initially inserted with the needles 20, 28. This may be advantageous for reducing the length of the repair suture 14 is utilized. Additionally, this may be advantageous for fully removing the shuttle suture 22 from the patient during the replacement procedure.

The pledget 12, as illustrated in FIGS. 4-6, may generally be rectangular or square in profile and may have a thin, generally flat body, referred to as the flat pledget 12A. In such configurations, the pledget 12A may be formed of a woven, mesh, or braided structure through which the repair suture 14 and the shuttle suture 22 extend and travel. The pledget 12 may be constructed of polytetrafluoroethylene, such as Teflon®, or may be constructed of a fiber material, such as polyester.

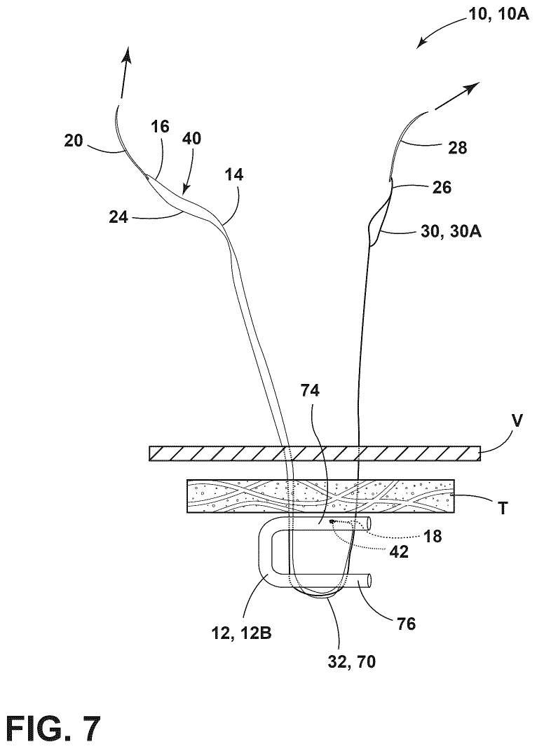

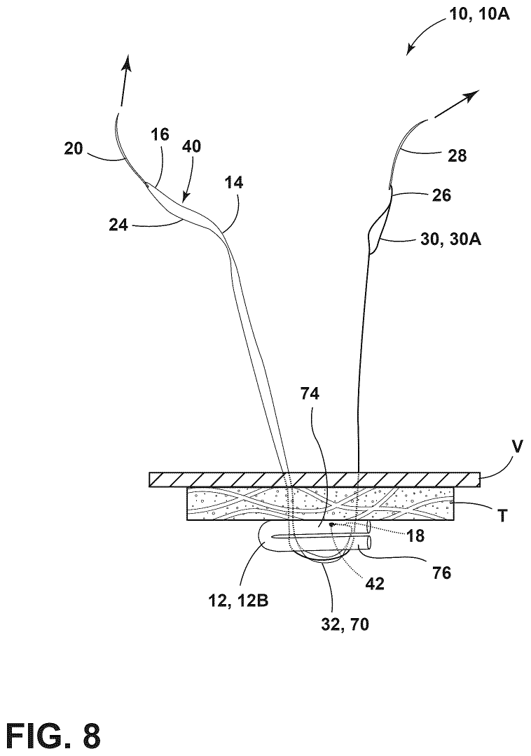

As illustrated in FIGS. 7 and 8, the pledget 12 may be a braided tubular component that can be folded, which may be referred to herein as a folded pledget 12B. The folded pledget 12B may be a braided tube that is folded to form more than one layer 74, 76. As in the illustrated configuration, the folded pledget 12B may include two layers 74, 76 with openings into the tubular structure being oriented in a same direction (illustrated as a right direction). Accordingly, the folded pledget 12B may form the first layer 74 which can engage the tissue T, and the second layer 76 which can engage the first layer 74. The folded pledget 12B may be constructed of polytetrafluoroethylene or polyester. Additionally, the folded pledget 12B may provide additional density and depth for forming the locking feature 32.

In configurations of the suture assembly 10 with the folded pledget 12B, the proximal end 18 of the repair suture 14 may be coupled to the first layer 74 via the base knot 42 proximate to a first end of the folded pledget 12B. The first end of the folded pledget 12B may be the end forming the openings into the tubular structure, as illustrated, or, alternatively, an end connecting the two layers 74, 76. The repair suture 14 may be configured to extend from the base knot 42, through the second layer 76 of the folded pledget 12B, along the second layer 76, through the second layer 76, through the first layer 74, and through the tissue T and the replacement valve V to the first needle 20.

The shuttle suture 22 may extend from the first needle 20, through the replacement valve V and the tissue T, through the first layer 74 proximate to the second end of the pledget 12B, through the second layer 76, along the second layer 76, through the second layer 76 proximate to the first end 24, through the first layer 74, and through the tissue T and the replacement valve V to the second needle 28. In certain aspects, the repair suture 14 and/or the shuttle suture 22 may not extend fully out of the second layer 76 and back into the second layer 76. In such examples, the repair suture 14 and/or the shuttle suture 22 may extend through the second layer 76 of the folded pledget 12B between the first and second ends of the folded pledget 12B, such as within a lumen defined by the folded pledget 12B.

In response to the needles 20, 28 being inserted through the tissue T and the replacement valve V, the sutures 14, 22 may be pulled to compress the folded pledget 12B for the second layer 76 to abut and press against the first layer 74, as illustrated in FIG. 8, in the configuration with the folded pledget 12B, the sutures 14, 22 may extend through a middle of the folded pledget 12B rather than the openings into the tubular structure. The openings are oriented in a first direction and the sutures 14, 22 may extend through the folded pledget 12B in a second direction, which may be normal to the first direction.

The suture assembly 10A illustrated in FIGS. 7 and 8 may be used in a substantially similar manner as described with respect to FIGS. 3-6. The needles 20, 28 may be removed from the sutures 14, 22, and the distal end 16 of the repair suture 14 may be inserted through the shuttle link 30A and folded to form the folded distal end 16. The pulling force may be applied to the second end 26 of the shuttle suture 22 to draw the shuttle link 30A and the folded distal end 16 through the tissue T and replacement valve V, through the pledget 12, and again through the tissue T and the replacement valve V to form the tensioned stitch.

Referring again to FIGS. 3-8, the suture assembly 10A may include the spliced section 70 in the repair suture 14, which may be in the pledget 12 or proximate to the pledget 12, such as along the second layer 76. The shuttle suture 22 may guide the repair suture 14 through itself at the spliced section 70 to lock the tensioned stitch as described herein. The locking feature 32 may correspond to the thickened portion of the spliced section 70 of the repair suture 14 that forms the locking trap as the repair suture 14 is drawn through itself. This configuration may be advantageous for reducing components in the suture assembly 10 and reducing components that remain in the patient. Further, this configuration may reduce or eliminate metal components, such as conventional crimps, from the suture assembly 10. The reduction or elimination of metal components may be advantageous for any subsequent X-ray or magnetic resonance imaging (MRI) where metal can affect the imaging process.

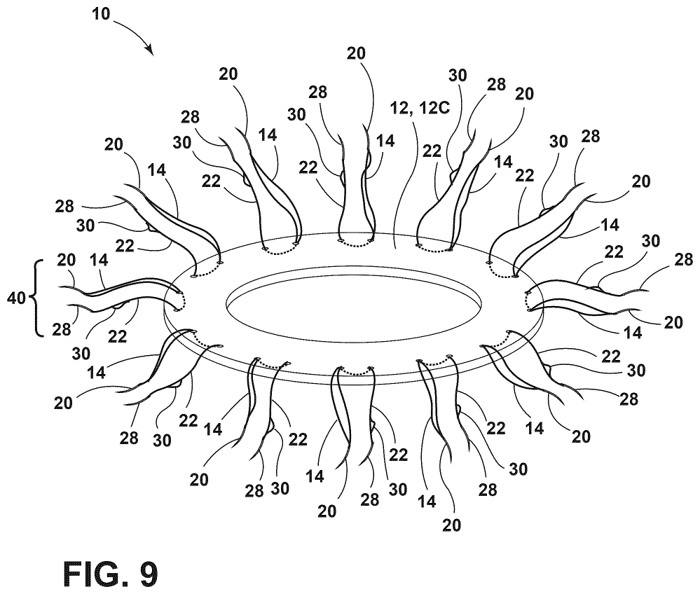

Referring to FIG. 9, in additional or alternative examples, the suture assembly 10 may include a ring-shaped or annular pledget 12C. The annular pledget 12C may be configured to be positioned around the annulus A. The suture assembly 10 may include suture pairs 40 to have multiple repair sutures 14 each with an associated shuttle suture 22. Accordingly, a single annular pledget 12C may be used with multiple repair sutures 14 and shuttle sutures 22 arranged in the suture pairs 40 (e.g., one repair suture 14 and one shuttle suture 22) along the ring formed by the pledget 12.

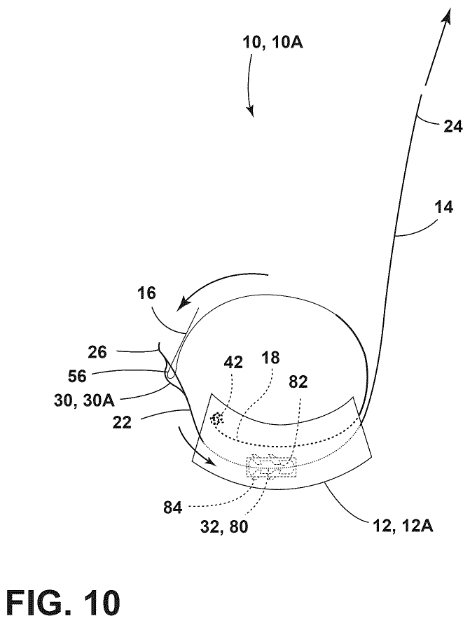

In this configuration, the annular pledget 12C may support most or all of the suture pairs 40 for the replacement valve procedure. For example, the annular pledget 12C may support between 12 and 17 suture pairs 40, which results in between 24 and 34 sutures 14, 22 and needles 20, 28. This configuration may be advantageous for maximizing efficiency when adding multiple suture pairs 40 around the annulus A during the valve replacement procedure. Additionally, this configuration may be advantageous for further distributing pressure along the annulus A from the tensioned sutures 14 and providing the compression between the tissue T and the replacement valve V. Each suture pair 40 may operate as described herein. Additionally, the suture assembly 10 may include multiple locking features 32, which may be in the form of the spliced section 70, a grommet 80 (see FIG. 10), or a combination thereof. The pledget 12 may also form different shapes or lengths that support different numbers of suture pairs 40 without departing from the teachings herein.

Referring to FIG. 10, in additional or alternative configurations, the locking feature 32 may be configured as a grommet 80 disposed in or coupled to the pledget 12. In certain aspects, the grommet 80 may be disposed in the mesh or braided material that forms the pledget 12. The grommet 80 may include inner projections or barbs 82 that may allow for the sutures 14, 22 to be passed in one direction and not the opposing direction. The inner barbs 82 may be angled to allow movement through the grommet 80 to tension the repair suture 14 and to reduce or prevent movement that would loosen the repair suture 14, locking the tensioned repair suture 14.

Additionally, the grommet 80 may include outer projections or barbs 84 that engage the braided structure of the pledget 12. This engagement may retain the grommet 80 in a predefined location relative to the pledget 12, including when the pulling force is applied to the sutures 14, 22 being pulled through the grommet 80. The fixed location of the grommet 80 generally assists with more fully tensioning the repair suture 14 and locking the repair suture 14 under tension.

In the configuration with the grommet 80, at least the shuttle suture 22 may initially extend through the grommet 80. The repair suture 14 may extend from the base knot 42 through the pledget 12 proximate to the grommet 80 or may extend through the grommet 80. As the shuttle suture 22 extends through the grommet 80, when the pulling force is applied to the shuttle suture 22, the shuttle link 30A may draw the folded distal end 16 of the repair suture 14 through the grommet 80 to then lock the repair suture 14 under tension.

While the grommet 80 is illustrated in FIG. 10 with the flat pledget 12A, the grommet 80 may be used in the tubular folded pledget 12B and/or the annular pledget 12C without departing from the teaching herein. In the tubular pledget 12B configuration, the grommet 80 may be disposed within the tubular structure (e.g., in an interior channel or lumen formed by the tube) or coupled to at least one of the layers 74, 76 of the folded pledget 12B.

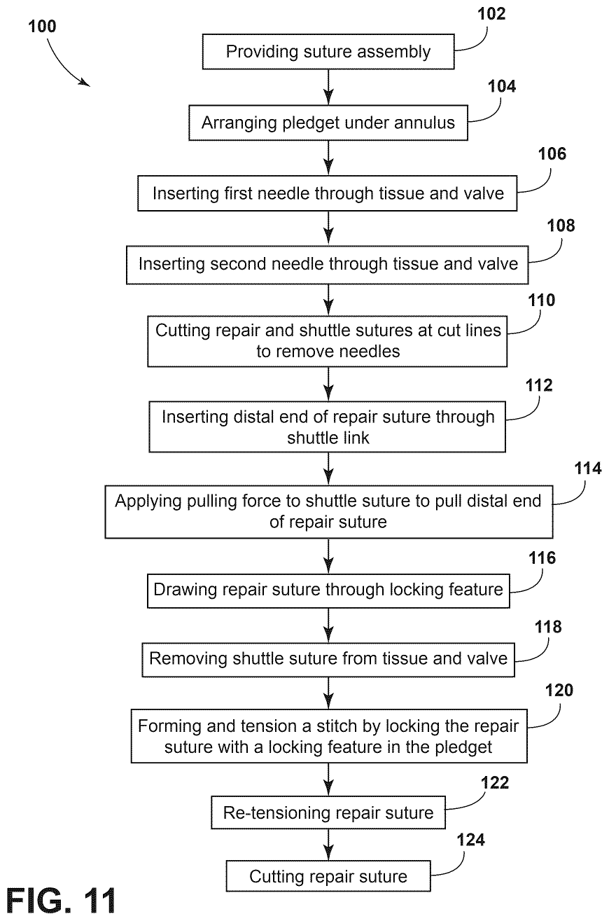

Referring to FIG. 11, and with further reference to FIGS. 1-10, a method 100 of using the suture assembly 10A for attaching an implant to tissue, such as in a valve replacement includes providing the suture assembly 10 (102) that includes the pledget 12, at least one repair suture 14 and associated shuttle suture 22, and the two needles 20, 28. As previously described, the leaflets of the aortic valve may be removed from the patient, and the replacement valve V may be provided. The pledget 12 can be arranged on the underside of the annulus A (104) where the replacement valve V is to be attached. The first needle 20 can be inserted through the tissue T and the replacement valve V (106), carrying the distal end 16 of the repair suture 14 and the first end 24 of the shuttle suture 22 through the tissue T and the replacement valve V.

The second needle 28 may be inserted through the tissue T and the replacement valve V (108), carrying the second end 26 of the shuttle suture 22 through the tissue T and the replacement valve V. At this stage, the ends of the sutures 14, 22 and the needles 20, 28 may be on an opposing side of the tissue T and the replacement valve V compared to the pledget 12. Tension may be applied by pulling both needles 20, 28 to move the pledget 12 to engage the tissue T.

The sutures 14, 22 may be cut (110) at the cut indicators 50-54 to remove the needles 20, 28 from the sutures 14, 22. The distal end 16 of the repair suture 14 and the first end 24 of the shuttle suture 22 may be separated from one another with the removal of the first needle 20. The distal end 16 of the repair suture 14 may be inserted or threaded through the shuttle link 30A (112). The distal end 16 may be folded at the fold indicator(s) 56 to form the folded distal end 16 around the shuttle link 30A.

The pulling force is applied to the first end 24 of the shuttle suture 22 (114), which may draw the folded distal end 16 of the repair suture 14 and the shuttle link 30A through the replacement valve V and the tissue T. The pulling force may draw the sutures 14, 22 into or through the pledget 12 and through the locking feature 32 (116). In configurations with the spliced section 70, the shuttle link 30A and the folded distal end 16 may be drawn through the spliced section 70. In such configurations, the repair suture 14 may be pulled through itself. The thickened portion of the repair suture 14 proximate to the spliced section 70 may bunch to lock the repair suture 14. In configurations with the grommet 80, the shuttle link 30A and the folded distal end 16 may be drawn through the grommet 80. The pulling force may continue to be applied to the shuttle suture 22 so the shuttle link 30A and the folded distal end 16 are drawn through the tissue T and the replacement valve V (118) proximate to an opposing side of the pledget 12. The shuttle suture 22 may be fully removed from the tissue T and the replacement valve V.

The repair suture 14 may continue to be pulled to form the stitch and tension the repair suture 14 (120). Additional suture assemblies 10 and/or suture pairs 40 may follow this process (e.g., steps 102-120 with multiple pledgets 12 or steps 106-120 with the annular pledget 12C) to attach the replacement valve V to the tissue T. One or more of the repair sutures 14 may be re-tensioned (122). An additional pulling force may be applied to the distal ends 18 of the repair sutures 14 to re-tension the repair sutures 14. The locking feature(s) 32 may automatically lock the repair sutures 14 with each re-tensioning.

A pusher/cutter device may be used to apply pressure on the replacement valve V while tensioning (120) and re-tensioning (122) the repair suture 14. This may be advantageous for reducing displacement of the replacement valve V and reducing slack from the suture assemblies 10. The repair sutures 14 may be cut (124) to be flush with the replacement valve V. The use of the pusher/cutter device may provide a better seal between the tissue T and the replacement valve V, as well as more fully tension the repair sutures 14 to then be cut flush with the replacement valve V. The locking features 32 retain the repair sutures 14 in the stitch configuration under tension. The steps 102-124 of the method 100 may be modified or conducted in different orders, with steps omitted, repeated, performed concurrently, etc. without departing from the teachings herein.

Referring to FIGS. 12 and 13, the suture assembly 10B is illustrated, which may provide an additional shuttling approach compared to the suture assembly 10A described in reference to FIGS. 1-11. The suture assembly 10B may include the pledget 12, the repair suture 14, the shuttle suture 22, and the locking feature 32. The shuttling approach provided by the suture assembly 10B may be based primarily on the configuration of the shuttle suture 22. Accordingly, the pledget 12, the repair suture 14, and the locking feature 32 may be substantially similar to any of the configurations described with reference to FIGS. 1-11.

The pledget 12, illustrated as the folded pledget 12B, may be configured to support the repair suture 14 and the shuttle suture 22. The pledget 12 in the suture assembly 10B may be any configuration, such as the flat pledget 12A, the folded pledget 12B, or the annular pledget 12C without departing from the teachings herein. Similar to the configuration described above, the proximal end 18 of the repair suture 14 may be coupled to the pledget 12 by the base knot 42. The repair suture 14 may have the increased thickness at or proximate to the proximal end 18, which may be from the stuffing 44. As previously set forth, the stuffing 44 may be additional suture material stuffed within the repair suture 14 to increase the width or thickness and assist with locking the repair suture 14. The repair suture 14 may extend through and out of the pledget 12 to the distal end 16. The distal end 16 of the repair suture 14 may taper in width, which may assist with drawing the repair suture 14 through the locking feature 32 and the pledget 12 during the shuttling process. The distal end 16 may be coupled with the first needle 20 for carrying the distal end 16 through the tissue T and the valve V.

As in the illustrated configuration of FIGS. 12 and 13, the locking feature 32 may be configured as the spliced section 70. At the spliced section 70, the repair suture 14 generally forms two limbs, and the shuttle suture 22 may be configured to extend between or be woven through the two limbs. This arrangement may allow the shuttle suture 22 to draw the distal end 16 of the repair suture 14 through the spliced section 70 of the repair suture 14. In response to the repair suture 14 being pulled through itself, the thicker part of the repair suture 14 with the stuffing 44 can bunch or catch to create the locking trap. The spliced section 70 and the bunched stuffing 44 may lock the repair suture 14 in the stitch configuration in response to tension applied to the opposing ends 16, 18. In this way, the repair suture 14 may be secured to retain the stitch configuration that couples the replacement valve V to the tissue T without requiring additional hardware or knots. Additionally, the grommet 80 (FIG. 10) may be similarly implemented to retain the repair suture 14 using the inner barbs 82.

The suture assembly 10B also includes the shuttle suture 22 for shuttling or guiding the repair suture 14, which may include the first end 24 coupled with the first needle 20 and the second end 26 coupled to the second needle 28. In this way, both the distal end 16 of the repair suture 14 and the first end 24 of the shuttle suture 22 may be coupled to the first needle 20. Additionally, both ends 24, 26 of the shuttle suture 22 may be coupled with needles 20, 28, respectively, for being carried through the tissue T and the implant/valve V. The first and second ends 24, 26 may be on opposing sides of the pledget 12 such that the middle section of the shuttle suture 22 extends through the pledget 12 and/or the locking feature 32.

Referring still to FIGS. 12 and 13, the shuttle suture 22 in the suture assembly 10B may be configured to shuttle or guide the repair suture 14 without including a specific component for receiving the repair suture 14. The shuttle suture 22 may include the shuttling feature 30, which may be configured as the shuttle receiving area 30B. The shuttle receiving area 30B may be one or more locations or regions on the shuttle suture 22 configured to receive the repair suture 14. Stated differently, the shuttle receiving area 30B may be an area or region through which the repair suture 14 is configured to be inserted for the shuttling process. In certain aspects, the shuttle receiving area 30B may be any location along the shuttle suture 22 between the pledget 12 and the second end 26 (e.g., location(s) on the shuttle suture 22 on an opposing side of the pledget 12 compared to the distal end 16 of the repair suture 14).

In additional or alternative examples, the shuttle receiving area 30B may be a predefined location or region that may maximize the efficiency of the shuttling process. In such examples, the shuttle receiving area 30B may be proximate to the second end 26 and the second needle 28, which may maximize the efficiency of the shuttling process and reduce excess length of the repair suture 14 in the pledget 12 in response to the repair suture 14 being tensioned. In the examples with a predefined location or region, the predefined location or region may be marked on the shuttle suture 22 with one or more identifiers 130 or other indicating marks.

In the configuration illustrated in FIGS. 12 and 13, the shuttle suture 22 may not have a preformed opening or loop through which the repair suture 14 is configured to be inserted. In this regard, the shuttle suture 22 may be a single limb from the first end 24 coupled with the first needle 20 to the second end 26 coupled with the second needle 28. This single limb may result in a substantially consistent thickness of the shuttle suture 22 from the first end 24 to the second end 26. The repair suture 14 may be configured to be inserted through the shuttle suture 22 at the shuttle receiving area 30B using a suture-through-suture approach. For example, when the shuttle suture 22 is formed by weaving or interlacing multiple yarns or strands of material, the repair suture 14 may be inserted between the different yarns or strands of material that form the shuttle suture 22. In certain aspects, the repair suture 14 may include one or more insertion indicators 132, which may be in addition to or in lieu of the fold indicator 56 (FIG. 3). The insertion indicator 132 may be proximate to the distal end 16 and provide a visual indication of how far to insert the repair suture 14 through the shuttle suture 22 for the shuttling process.

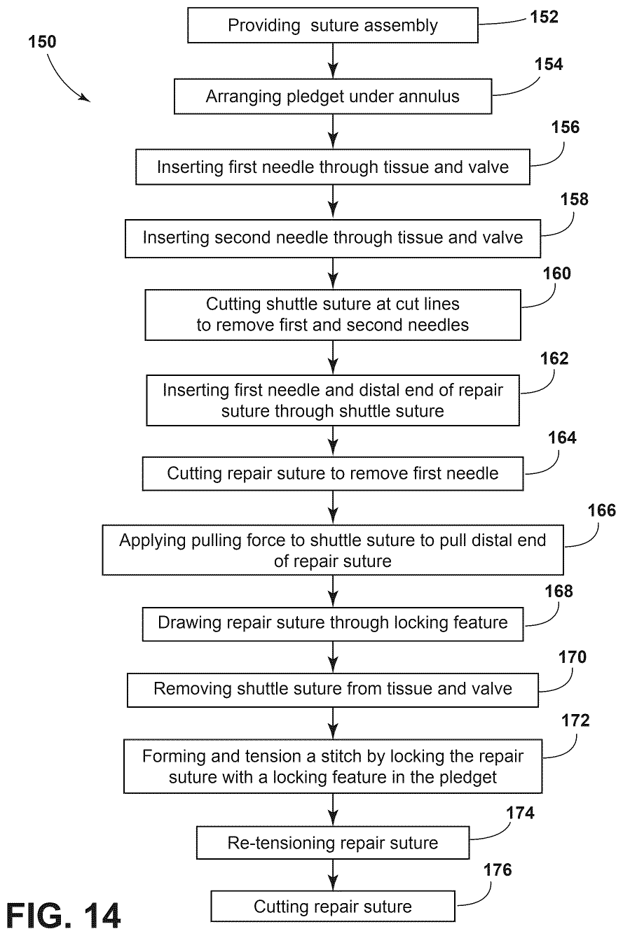

Referring to FIG. 14, and with further reference to FIGS. 12 and 13, the suture assembly 10B may form the stitch and can be used in a valve replacement procedure or other implant attachment procedure in a substantially similar manner as the suture assembly 10A, with differences primarily in the shuttling approach. A method 150 of using the suture assembly 10B for a valve replacement may include providing the suture assembly 10B (152) that includes the pledget 12, at least one repair suture 14 and associated shuttle suture 22, and the two needles 20, 28. As previously set forth, the leaflets of the aortic valve may be removed from the patient, and the replacement valve V may be provided. The pledget 12 can be arranged on the underside of the annulus A (154) where the replacement valve V is attached.

The first needle 20 can be inserted through the tissue T and the replacement valve V (156). With the repair suture 14 and the shuttle suture 22 coupled to the first needle 20, the first needle 20 may carry the distal end 16 of the repair suture 14 and the first end 24 of the shuttle suture 22 through the tissue T and the replacement valve V. Similarly, the second needle 28 may be inserted through the tissue T and the replacement valve V (158). The second needle 28 can carry the second end 26 of the shuttle suture 22 through the tissue T and the replacement valve V.

In response to steps 156 and 158, the ends 18, 24, 26 of the sutures 14, 22 and the needles 20, 28 may be on the opposing side of the tissue T and the replacement valve V (e.g., proximate an outer surface of the replacement valve V) compared to the pledget 12 (e.g., abutting an inner surface of the tissue T). Tension may be applied by pulling both needles 20, 28 to apply tension to the sutures 14, 22 to move the pledget 12 to engage the tissue T. The sutures 14, 22 may cause an initial engagement between the tissue T and the valve V, with the ends 18, 24, 26 arranged to prepare for the shuttling process to form the seal between the tissue T and the valve V.

With the ends 18, 24, 26 exposed on an outside of the tissue T and the valve V, the shuttle suture 22 may be cut (160) at the cut indicators 52, 54 to remove the needles 20, 28 from the shuttle suture 22. However, the distal end 16 of the repair suture 14 may remain coupled with the first needle 20. Accordingly, the distal end 16 of the repair suture 14 and the first end 24 of the shuttle suture 22 may be separated from one another by cutting the shuttle suture 22.

At least the second end 26 of the shuttle suture 22 may be held to move the first needle 20 and, consequently, the distal end 16 of the repair suture 14 relative to the shuttle suture 22. The first needle 20 may be inserted or threaded through the shuttle suture 22 (162), which brings the distal end 16 of the repair suture 14 through the shuttle suture 22, forming the suture-through-suture configuration. The repair suture 14 may be inserted through the shuttle receiving area 30B of the shuttle suture 22, which is generally proximate to the second end 26. The repair suture 14 may be pulled through the shuttle suture 22 such that the distal end 16 and an adjacent section (e.g., an exposed length outside of the pledget 12) of the repair suture 14 are on opposing sides of the shuttle suture 22. In certain aspects, the insertion indicator 132 may be used as a guide for how far to pull the repair suture 14 through the shuttle suture 22 to maximize the efficiency of the shuttling process.

With the repair suture 14 extending through the shuttle suture 22, the repair suture 14 may be cut (164) at the cut indicator 50 to remove the first needle 20 from the distal end 16. Accordingly, the first and second needles 20, 28 may be removed from the suture assembly 10B for the shuttling process. For the shuttling process, the pulling force can be applied to the first end 24 of the shuttle suture 22 (166), which may draw the shuttle receiving area 30B of the shuttle suture 22 and, consequently, the distal end 16 of the repair suture 14 through the replacement valve V and the tissue T. The pulling force may draw the sutures 14, 22 into or through the pledget 12 and through the locking feature 32 (168).

In configurations with the spliced section 70, the shuttle receiving area 30B and the distal end 16 may be drawn through the spliced section 70 to pull the repair suture 14 through itself. The thickened portion of the repair suture 14 (e.g., with the stuffing 44) proximate to the spliced section 70 may bunch, forming the locking trap to lock the repair suture 14 in the looped or stitch configuration. In configurations with the grommet 80, the shuttle receiving area 30B and the distal end 16 may be drawn through the grommet 80. The inner barbs 82 may allow movement of the sutures 14, 22 in one direction (e.g., the pulling or tensioning direction) and reduce or prevent movement in the opposing direction (e.g., a loosening direction) to lock the repair suture 14 in the stitch configuration.

The tension or pulling force may continue to be applied to the shuttle suture 22 until the shuttle receiving area 30B and the distal end 16 are drawn through the locking feature 32, the tissue T, and the replacement valve V (170) proximate to an opposing side of the pledget 12. The shuttle suture 22 may be fully removed from the tissue T and the replacement valve V. The repair suture 14 may form the stitch or loop that extends through the tissue T and the replacement valve V three times (e.g., once from the first needle 20 and twice from the shuttling process).

The distal end 16 of the repair suture 14 may continue to be pulled to form the stitch and tension the repair suture 14 (172). Additional suture assemblies 10 and/or suture pairs 40 may follow this process (e.g., steps 152-172 with multiple pledgets 12 or steps 156-172 with the annular pledget 12C) to attach the replacement valve V to the tissue T. One or more of the repair sutures 14 may be re-tensioned (174), which may be advantageous for initially attaching the valve V to the tissue T and then more fully forming the seal therebetween. Often, the replacement valve V may shift or move during the replacement procedure. The ability of the suture pairs 40 to be re-tensioned may allow for more accurate alignment of the valve V to be finalized once each of the suture pairs 40 has been initially tensioned. The locking features 32 may automatically lock the repair sutures 14 with each re-tensioning.

As previously noted, the pusher/cutter device may be used to apply pressure on the replacement valve V while tensioning (172) and re-tensioning (174) the repair suture 14. This may assist with aligning the valve V and forming the fluid-tight seal. In response to the tensioning and re-tensioning, the repair sutures 14 may be cut (176) to be flush with the replacement valve V, removing excess length of the repair suture 14. The locking features 32 retain the repair sutures 14 in the stitch configuration under tension. The steps 152-176 of the method 150 may be modified or conducted in different orders, with steps omitted, repeated, performed concurrently, etc. without departing from the teachings herein.

The suture assembly 10B may provide an efficient shuttling approach with the suture-through-suture process. Additionally, the suture assembly 10B may provide a smaller overall footprint with the shuttle suture 22 may be smaller/thinner than the shuttle suture 22 used in the suture assembly 10A. Typically, sutures 22 that are bifurcated or have a bifurcated segment have a sufficient thickness or a sufficient number of strands/yarns for the manufacturing machine to form the two limbs. The use of the shuttle receiving area 30B may allow for a single limb along the length of the shuttle suture 22 that is smaller/thinner than when the shuttle link 30A is used.

With reference now to FIGS. 15 and 16, the suture assembly 10C is illustrated, which may function to provide the stitch configuration with a different shuttling approach compared to the suture assembly 10A described in reference to FIGS. 1-11 and the suture assembly 10B described in reference to FIGS. 12-14. The suture assembly 10C may include the pledget 12, the repair suture 14, the shuttle suture 22, and the locking feature 32. The different shuttling approach provided by the suture assembly 10C may be based primarily on the configuration and arrangement of the shuttle suture 22. Accordingly, the pledget 12, the repair suture 14, and the locking feature 32 may be substantially similar to any of the configurations described as described with reference to FIGS. 1-14.

Similar to the prior suture assembly 10A, 10B configurations, in the suture assembly 10C, the pledget 12 may support the sutures 14, 22. The pledget 12 is illustrated as the folded pledget 12B but may be any configuration for supporting the sutures 14, 22 without departing from the teachings herein. The proximal end 18 of the repair suture 14 may be coupled to the pledget 12 by the base knot 42, and the repair suture 14 may extend through and out of the pledget 12. The distal end 16 of the repair suture 14 may have the tapered width and may be coupled with the first needle 20 for being carried through the tissue T and the valve V. The tapered distal end 16 may assist with the shuttling process, reducing friction or resistance as the distal end 16 is drawn or pulled through the pledget 12, the locking feature 32, the valve V, and the tissue T.

The proximal end 18 of the repair suture 14 may have the increased thickness with the stuffing 44, which may be additional suture material stuffed within the repair suture 14 proximate to the proximal end 18 to increase the width or thickness to assist with locking the repair suture 14. The stuffing 44 and the thickened proximal end 18 may be configured to bunch in response to the shuttling process, forming the locking trap for retaining the repair suture 14 in the stitch configuration. As in the illustrated configuration, the locking feature 32 may be configured as the spliced section 70 with the repair suture 14 extending through the spliced section 70.

The shuttle suture 22 may be configured to guide or shuttle the repair suture 14 through the spliced section 70 during the shuttling process, which may cause the stuffing 44 to bunch in response to tensioning or re-tensioning the repair suture 14 to lock the repair suture 14 in the stitch configuration. It is also contemplated that the grommet 80 may additionally or alternatively be used as the locking feature 32 without departing from the teachings herein. In such examples, the inner barbs 82 may allow the sutures 14, 22 to be moved through the locking feature 32 in the pulling/tensioning direction and reduce or prevent movement of the sutures 14, 22 in the opposing direction to lock the tensioned repair suture 14 in the stitch configuration.

In the suture assembly 10C illustrated in FIGS. 15 and 16, the shuttle suture 22 may be folded to form the shuttling feature 30, which may be configured as the folded shuttle loop 30C for use in the shuttling process. In certain aspects, the shuttle suture 22 may be folded at a general midpoint, mid-region, or mid-section 200 between the first and second ends 24, 26 to form two limbs. The folded shuttle suture 22 may extend through the pledget 12.

Accordingly, in this configuration, the first and second ends 24, 26 of the shuttle suture 22 may be disposed adjacent to one another, and the ends 24, 26 may be spaced from the mid-section 200 by the pledget 12. In this regard, both limbs of the folded shuttle suture 22 may extend through the pledget 12, with a first limb extending between the first end 24 and the mid-section 200 and the second limb extending between the second end 26 and the mid-section 200. A section of the first limb between the first end 24 and the mid-section 200 may extend through the pledget 12 adjacent to a section of the second limb between the second end 26 and the mid-section 200. Generally, both limbs may engage the pledget 12 and/or the locking feature 32 in a same or similar location for shuttling or guiding the repair suture 14 and for removing the shuttle suture 22 from the pledget 12 after the stitch is formed.

To utilize the suture assembly 10C to couple the implant or valve V with the tissue T and form the fluid-tight seal, the sutures 14, 22 may be coupled with the needles 20, 28 for carrying the sutures 14, 22 through the tissue T and the valve V. In certain aspects, the first and second end 24, 26 of the shuttle suture 22 may be disposed adjacent to the distal end 16 of the repair suture 14. Each of the ends 18, 24, 26 may be coupled with the first needle 20 to allow the ends 18, 24, 26 to be carried through the tissue T and the valve V concurrently. The ends 18, 24, 26 may be separately coupled to the needle 20 or may combined or spliced together to reduce the number of tails coupled to the needle 20.

The mid-section 200 of the shuttle suture 22 may be carried through the tissue T and the valve V separately from the ends 24, 26, generally using the second needle 28. The folded shuttle loop 30C may be coupled with the second needle 28 via a connecting suture 202. The connecting suture 202 may form a loop through the folded shuttle loop 30C and have both ends or tails coupled to the second needle 28, consequently coupling the folded shuttle loop 30C with the second needle 28. The connecting suture 202 may be constructed similarly to the repair suture 14 and/or the shuttle suture 22. The second needle 28 may carry the connecting suture 202 and the folded shuttle loop 30C through the tissue T and the valve V. The needles 20, 28 may be utilized to arrange the pledget 12 adjacent to the inner surface of the tissue T with the ends 18, 24, 26 and the mid-section 200 on an opposing side of the tissue T and the valve V.

With the ends 18, 24, 26 and the mid-section 200 carried through the tissue T and the valve V, the needles 20, 28 may be removed for the shuttling process. The folded shuttle loop 30C may be formed by the two limbs between the pledget 12 and the mid-section 200 (e.g., the middle section of the shuttle suture 22 forms the folded shuttle loop 30C). This may provide a larger insertion location for the repair suture 14 for the shuttling process. The distal end 16 of the repair suture 14 may be inserted through the folded shuttle loop 30C and folded, such as at the fold indicator 56. This engagement may allow the shuttle suture 22 to carry or shuttle the repair suture 14 to form the stitch.

Tension may be applied to both ends 24, 26 of the shuttle suture 22. In response, the folded shuttle loop 30C can be drawn through the pledget 12 and the locking feature 32. As the folded shuttle loop 30C is pulled by the tension or pulling force, the folded shuttle loop 30C may also pull the distal end 16 of the repair suture 14 through the pledget 12 and the locking feature 32. The locking feature 32 may be configured to lock the repair suture 14 in the looped or stitch configuration, such as by bunching the stuffing 44 when the locking feature 32 is the spliced section 70 or with the inner barbs 82 when the locking feature 32 is the grommet 80. In the stitch configuration, the full extension of the repair suture 14 may be: from the base knot 42, through the pledget 12, through the tissue T, through the valve V, across the valve V, through the valve V, through the tissue T, through the locking feature 32, through the tissue T, and through the valve V. Accordingly, the repair suture 14 may attach and form a seal between the valve V to the tissue T.

The folded suture 22 may result in two limbs of the shuttle suture 22 being carried through the tissue T and valve V concurrently. To utilize the folded shuttle suture 22, the shuttle suture 22 may be a smaller suture compared to at least the shuttle suture 22 with the bifurcated shuttle link 30A in the suture assembly 10A (see FIGS. 3-10). It is also contemplated that the folded shuttle suture 22 in this configuration of the suture assembly 10C may be a smaller suture 22 than the shuttle suture 22 with the shuttle receiving area 30B in the suture assembly 10B (see FIGS. 12 and 13). The smaller shuttle suture 22 in the suture assembly 10C may allow the shuttle suture 22 to be folded while maintaining a low profile or smaller overall suture assembly 10C. In certain aspects, the shuttle suture 22 may be a monofilament suture. Additionally or alternatively, the shuttle suture 22 may be a 4-0 or 5-0 suture, such as, for example, a 4-0 or 5-0 Prolene® suture. The smaller shuttle suture 22 may allow the suture 22 to be folded to form the folded shuttle loop 30C and to reduce the overall footprint of the suture assembly 10C.

The configuration and size of the shuttle suture 22 may assist with providing a large enough shuttle suture 22 to receive and guide the repair suture 14 while maintaining a low profile overall assembly 10. The folded shuttle suture 22 may also increase manufacturing efficiency. For example, forming bifurcated sutures 22 can increase complexity in the manufacturing process, as well as increase the size of the shuttle suture 22 being used. A certain number of yarns or strands of material may typically be utilized to form bifurcated suture segments, which can increase the size of the suture 22.

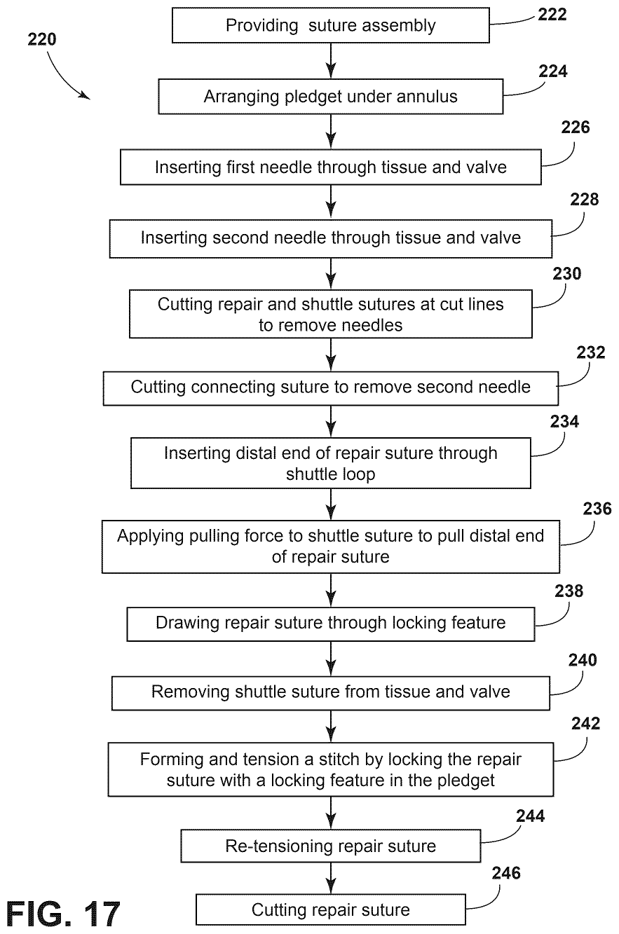

Referring to FIG. 17, as well as FIGS. 14 and 15, the suture assembly 10C may form the stitch and can be used in a valve replacement procedure or other implant attachment procedure in a substantially similar manner as the suture assemblies 10A, 10B, with differences primarily in the shuttling approach and how the shuttle suture 22 is inserted through the tissue T and the valve V with the needles 20, 28. A method 220 of using the suture assembly 10C for a valve replacement may include providing the suture assembly 10C (222) that includes the pledget 12, at least one repair suture 14 and associated shuttle suture 22, and the two needles 20, 28. As previously noted, the leaflets may be removed from the patient, and the replacement valve V may be provided. The pledget 12 can be arranged on the underside of the annulus A (224).

The first needle 20 can be inserted through the tissue T and the replacement valve V (226). The first needle 20 may carry the distal end 16 of the repair suture 14 and both the first end 24 and the second end 26 of the shuttle suture 22 through the tissue T and the replacement valve V. Similarly, the second needle 28 may be inserted through the tissue T and the replacement valve V (228). The second needle 28 may carry the connecting suture 202 and the folded shuttle loop 30C of the shuttle suture 22 through the tissue T and the replacement valve V. At this stage, the ends 18, 24, 26 of the sutures 14, 22, the folded shuttle loop 30C (e.g., the mid-section 200 of the shuttle suture 22), and the needles 20, 28 may be on the opposing side of the tissue T and the replacement valve V compared to the pledget 12. Further, the ends 18, 24, 26 of the sutures 14, 22 may generally be spaced from the folded shuttle loop 30C, which can allow the repair suture 14 to extend over the valve V to form the stitch as a result of the shuttling process. Tension may be applied by pulling both needles 20, 28 to move the pledget 12 to engage the tissue T and reduce excess suture 14, 22 length under the tissue T and/or valve V.

With the ends 18, 24, 26 and the folded shuttle loop 30C carried through the tissue T and the valve V, the sutures 14, 22 may be cut (230) at the cut indicators 50-54 to remove the first needle 20 from the ends 18, 24, 26 of the sutures 14, 22 for the shuttling process. With the removal of the first needle 20, the distal end 16 of the repair suture 14 and the ends 24, 26 of the shuttle suture 22 may be separated from one another. The connecting suture 202 may be cut (232) to remove the second needle 28 and the connecting suture 202 from the folded shuttle loop 30C of the shuttle suture 22.

The distal end 16 of the repair suture 14 may be inserted or threaded through the folded shuttle loop 30C (234) for the shuttling process. The distal end 16 may be folded at the fold indicator(s) 56 to form the folded distal end 16 around the folded shuttle loop 30C. The pulling force is applied to the ends 24, 26 of the shuttle suture 22 (236), which may draw the folded shuttle loop 30C with the folded distal end 16 of the repair suture 14 through the replacement valve V and the tissue T. The pulling force may draw the sutures 14, 22 into or through the pledget 12 and through the locking feature 32 (238). In certain aspects, the folded shuttle loop 30C and the folded distal end 16 may be drawn through the spliced section 70 to pull the repair suture 14 through itself, bunching the stuffing 44 and forming the locking trap to lock the repair suture 14 in the stitch configuration. Additionally or alternatively, the folded shuttle loop 30C and the folded distal end 16 may be drawn through the grommet 80, with the inner barbs 82 locking the repair suture 14.

The pulling force may continue to be applied to the shuttle suture 22 until the folded shuttle loop 30C and the folded distal end 16 are drawn through the tissue T and the replacement valve V (240) proximate to an opposing side of the pledget 12 (e.g., after being pulled through the locking feature 32). With the shuttling process, the shuttle suture 22 may be fully removed from the tissue T and the replacement valve V. Further, with the shuttle suture 22 removed, the repair suture 14 generally remains in the stitch or loop configuration, connecting the valve V to the tissue T.

To form the seal between the tissue T and the valve V, the repair suture 14 may continue to be pulled to form the stitch and tension the repair suture 14 (242). Additional suture assemblies 10 and/or suture pairs 40 may follow this process (e.g., steps 222-242 with multiple pledgets 12 or steps 226-242 with the annular pledget 12C) to attach the replacement valve V to the tissue T. As the suture pairs 40 are tensioned, the valve V may move in response to the tension forces being applied. The valve V may be moved after an initial tensioning process, and one or more of the repair sutures 14 may be re-tensioned (244) to more fully form the seal between the tissue T and the valve V. Additional pulling force may be applied to the distal ends 18 of the repair sutures 14 to re-tension the repair sutures 14, and the locking features 32 may automatically lock the repair sutures 14 with each re-tensioning.

As previously noted, the pusher/cutter device may be used to apply pressure on the replacement valve V while tensioning (242) and re-tensioning (244) the repair suture 14. This may be advantageous for reducing displacement of the replacement valve V and reducing slack from the suture assemblies 10C. The repair suture 14 may be cut (246) to be flush with the replacement valve V, removing excess length from the repair suture 14 from the surgical site. The locking features 32 retain the repair sutures 14 in the stitch configuration under tension. The steps 222-246 of the method 220 may be modified or conducted in different orders, with steps omitted, repeated, performed concurrently, etc. without departing from the teachings herein.

Referring again to FIGS. 3-17, the suture assemblies 10A, 10B, 10C may include the repair suture 14 fixed to the pledget 12 and the shuttle suture 22. Each of the shuttle suture 22 and the repair suture 14 may be coupled to one or both of the needles 20, 28 for carrying the suture assembly 10 through the tissue T and the replacement valve V or other implants. Accordingly, with a single pass-through of each needle 20, 28, one or both of the repair suture 14 and the shuttle suture 22 may be carried through the tissue T and the valve V to position the pledget 12 against the tissue T and make the ends 16, 18, 24, 26 available on an outer side of the valve V for the shuttling process.

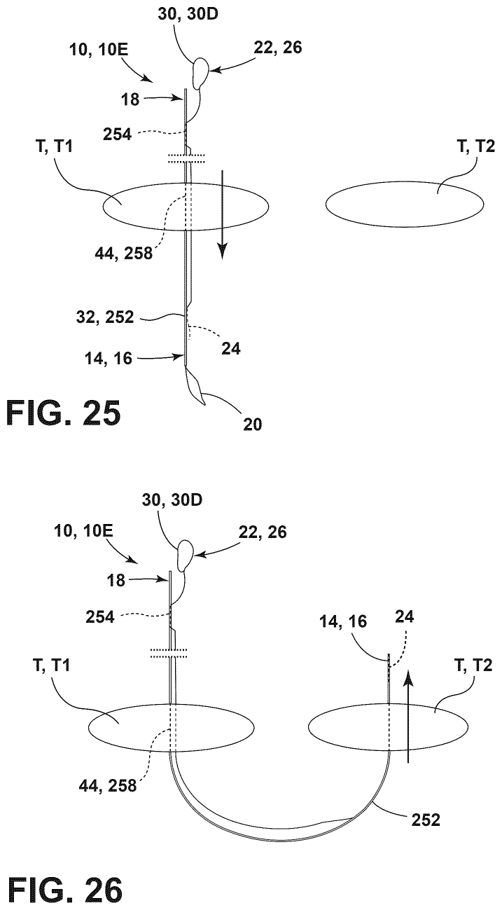

With reference to FIGS. 18-22, additional exemplary configurations of the knotless suture assembly 10 are illustrated, including suture assemblies 10D, 10E. The suture assemblies 10D, 10E may utilize the repair suture 14 and the shuttle suture 22. The repair suture 14 may be utilized to attach implants, such as the replacement valve V, to the tissue T, as described with respect to FIGS. 1-17, or to connect multiple tissue bodies T1, T2 together, as will be described further herein with respect to FIGS. 25-38. The shuttle suture 22 may be utilized to shuttle or guide the repair suture 14 to form the stitch configuration in response to the pulling force or tension applied to the shuttle suture 22, similar to the process described above with respect to FIGS. 4-6. Sections of the repair suture 14 and the shuttle suture 22 may be spliced or otherwise combined together during a manufacturing process for efficient insertion during a surgical procedure and may be configured to be at least partially separated during the procedure to proceed with the shuttling process and subsequent removal of the shuttle suture 22.

Referring to FIGS. 18 and 19, in this configuration of the suture assembly 10D, the suture assembly 10D may include the repair suture 14 having a substantially similar length as the shuttle suture 22. The first end 16 of the repair suture 14 may be arranged adjacent to the first end 24 of the shuttle suture 22, and the second end 18 of the repair suture 14 may be disposed adjacent to the second end 26 of the shuttle suture 22. In other words, the second end 18 of the repair suture 14 may not be fixed.

The suture assembly 10D may include one or more interlacing or spliced segments 252, 254, 256 that couple the repair suture 14 and the shuttle suture 22. At these segments 252, 254, 256 the shuttle suture 22 may be spliced into, combined with, embedded in, encapsulated in, interlaced with, or otherwise joined with the repair suture 14. Typically, the shuttle suture 22 may be spliced into the repair suture 14; however, the repair suture 14 may be spliced into the shuttle suture 22 without departing from the teachings herein. The spliced segments 252, 254, 256 may allow the sutures 14, 22 to be interlaced together forming an integrated suture assembly 10D that can maximize the efficiency of the procedure. For example, the integrated suture assembly 10D may be inserted into/through the tissue T as a single body, and the sutures 14, 22 may then be at least partially separated to move independently of one another for the shuttling process.

The repair suture 14 may include the first end 16, which may be referred to herein as a lead or movable end 16, and the second opposing end 18, which may be referred to herein as a static end 18. The repair suture 14 may have a predefined length between the first and second ends 16, 18. The predefined length may depend on the patient anatomy or procedure for which the suture assembly 10 is being used. One or both of the ends 16, 18 may be tapered to assist with inserting the suture assembly 10 through the tissue T. The shuttle suture 22 may include the first end 24, which may be referred to as the non-looped end 24, and the second opposing end 26, which may be referred to as a looped end 26. The looped end 26 may be arranged proximate to the static end 18 of the repair suture 14, and the non-looped end 24 may be arranged proximate to the lead end 16 of the repair suture 14.

The second end 26 of the shuttle suture 22 may define or include the end shuttle loop 30D, which may be an opening formed from folding the suture 22. The end 26 of the shuttle suture 22 may be folded to engage itself to form the end loop 30D. In this configuration, the width of the shuttle suture 22 at the end loop 30D may be greater than the width of the remainder of the shuttle suture 22. Alternatively, the end 26 may be tapered to reduce the thickness of the loop 30D. This configuration may be advantageous for maximizing the efficiency of the manufacturing process. The shuttle loop 30D may also be formed through a bifurcation process. In bifurcated examples, during the manufacturing process, the shuttle suture 22 may be formed of interlacing strands as a single limb, then formed into two limbs to form the bifurcated segment, and then again formed as a single limb to form the shutting feature 30, similar to the shuttle link 30A described with respect to FIGS. 3-8. This process may be advantageous for retaining a smaller width of the shuttle suture 22 at the shuttle end loop 30D.

The repair suture 14 and the shuttle suture 22 may be spliced or otherwise combined together in segments 252, 254, 256 at multiple locations along the length of the repair suture 14 to form the combined or integrated suture assembly 10D. The suture assembly 10D may be manufactured to have the preformed spliced segments 252, 254, 256 and allow the integrated suture assembly 10 to be readily unpackaged and used in a sterile setting. The preformed engagement or spliced segments 252, 254, 256 may be advantageous for maximizing the efficiency of the procedure and reducing separate lengths of the sutures 14, 22 that may become tangled during the procedure.

At the first spliced segment 252, the first end 24 of the shuttle suture 22 may be spliced into the repair suture 14 at or proximate to the first end 16 of the repair suture 14 (e.g., at a first location). In certain aspects, the repair suture 14 may extend a predefined length beyond the first end 24 of the shuttle suture 22. The predefined length may be minimal such that the ends 16, 24 may be adjacent to one another or may be greater such that a portion of the repair suture 14 extends beyond the end 24 of the shuttle suture 22 that is combined, joined, embedded, or otherwise spliced into the repair suture 14. By splicing the end 24 of the shuttle suture 22 into the repair suture 14, the end of the suture assembly 10 may have a single tail.

The shuttle suture 22 may be spliced into the repair suture 14 at a second location forming the second spliced segment 254, t. For example, the second end 26 of the shuttle suture 22 may be spliced into the repair suture 14 at or proximate to the second end 18. Accordingly, the shuttle loop 30D may be spliced into the repair suture 14. Similar to the first ends 16, 24, the repair suture 14 may extend a predefined length beyond the second end 26 of the shuttle suture 22. This predefined length may be minimal such that the ends 18, 26 may be adjacent to one another or may be greater such that a portion of the repair suture 14 extends beyond the end 26 of the shuttle suture 22 that is combined, joined, embedded, or otherwise spliced into the repair suture 14. By splicing the end 26 of the shuttle suture 22 into the repair suture 14, both ends of the suture assembly 10 may have a single tail, which can subsequently be separated after insertion through the tissue T for the shuttling process.

The shuttle suture 22 may be spliced into the repair suture 14 at a third location. The third spliced segment 256 may be between the first and second spliced segments 252, 254 and may be generally aligned with the mid-section 200 and a mid-region 258 of one or both of the repair suture 14 and the shuttle suture 22. The third spliced segment 256 may form the locking feature 32 of the suture assembly 10 (similar to the spliced section 70 described above). The third spliced segment 256 may allow the shuttle suture 22 to move relative to the repair suture 14 to, ultimately, move through and out of the repair suture 14 during the shuttling process.

In various aspects, additional stuffing 44 may be spliced or otherwise integrated into the repair suture 14, which may be at a spliced region 262. In the illustrated example, the stuffing 44, which may also be referred to as a stuffing suture 44, may be added at the spliced region 262 between the first spliced segment 252 and the third spliced segment 256. Typically, the stuffing 44 may be adjacent to the third spliced segment 256 (e.g., the locking feature 32) on an opposite side of the shuttle loop 30D. During the shuttling process, the repair suture 14 may be drawn through the third spliced segment 256 and toward the stuffing 44, which may cause the stuffing 44 to bunch and lock the repair suture 14 in the looped, stitch configuration.

The ends of the suture assembly 10D may be coupled with the needles 20, 28, respectively. As the shuttle suture 22 may be spliced into the repair suture 14, the first and second ends 16, 18 of the repair suture 14 may be coupled with the needles 20, 28, respectively, which can carry the ends 24, 26 of the shuttle suture 22 through the tissue T. Accordingly, the suture assembly 10D may include the two needles 20, 28 for carrying the suture assembly 10D through the tissue T and/or the implant/valve V in a manner substantially similar as described with respect to FIGS. 3-8. The ends 24, 26 of the shuttle suture 22 being spliced into the repair suture 14 may reduce resistance or friction as the suture assembly 10D is carried through the tissue T and/or valve V. Additionally, a single suture body (e.g., the repair suture 14 or the combined repair suture 14 and shuttle suture 22) may initially be inserted through the tissue T with the needles 20, 28, which may assist with a smoother insertion process.

After the needles 20, 28 have carried the suture assembly 10 through the tissue T and/or the valve V, the ends of the suture assembly 10 may be cut. The suture assembly 10D may have cut indicators 52, 54 to identify an optimal location for cutting the suture assembly 10D. Cutting the suture assembly 10D may separate the ends 16, 18 of the repair suture 14 from the ends 24, 26 of the shuttle suture 22. Accordingly, the suture assembly 10D may be cut between the middle spliced segment 256 and each of the end spliced segments 252, 254. Alternatively, cutting at the spliced segments 252, 254 may allow the sutures 14, 22 to be separated.

Accordingly, the sutures 14, 22 may be joined or spliced together for insertion through the tissue T and then separated with the removal of the needles 20, 28 for the shuttling process. In this way, cutting the needles 20, 28 from the suture assembly 10 may expose the shuttle loop 30D and separate the movable end 16 of the repair suture 14 from the non-looped end 24 of the shuttle suture 22. The movable end 16 may be inserted through the shuttle loop 30D, folded, and carried through the middle spliced segment 256, similar to the process described with respect to FIG. 4-6. The locking feature 32, which may include the third spliced segment 256 and the stuffing 44, may lock the repair suture 14 in the stitch configuration.

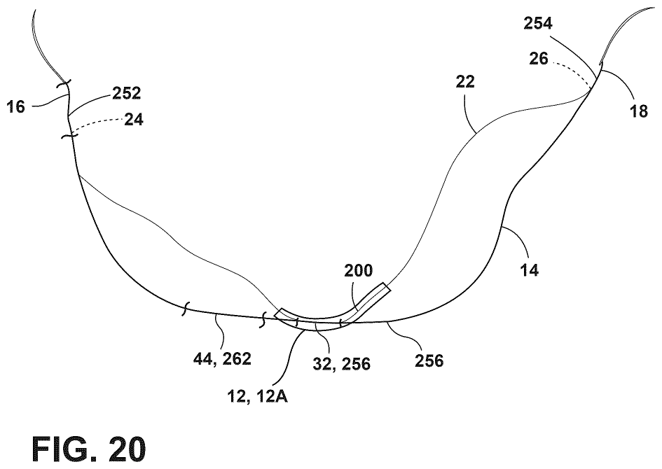

As illustrated in FIGS. 18 and 19, the suture assembly 10D may not include the pledget 12. Such configurations may be advantageous for more orthopedic approaches and procedures, such as connecting tissue bodies T1, T2 together. In additional or alternative exampled, such as illustrated in FIG. 20, the suture assembly 10D may include the pledget 12. The suture assembly 10D may exit from different points on the pledget 12 to create a buttress for anchoring in the tissue T. In certain aspects, the repair suture 14 and the shuttle suture 22 may each exit the pledget 12 at the same two opposing points or at two different opposing points. The suture assembly 10D may extend from opposing sides of the pledget 12, which may have any configuration described herein. The pledget 12 may assist in distributing the force applied by the suture assembly 10D.

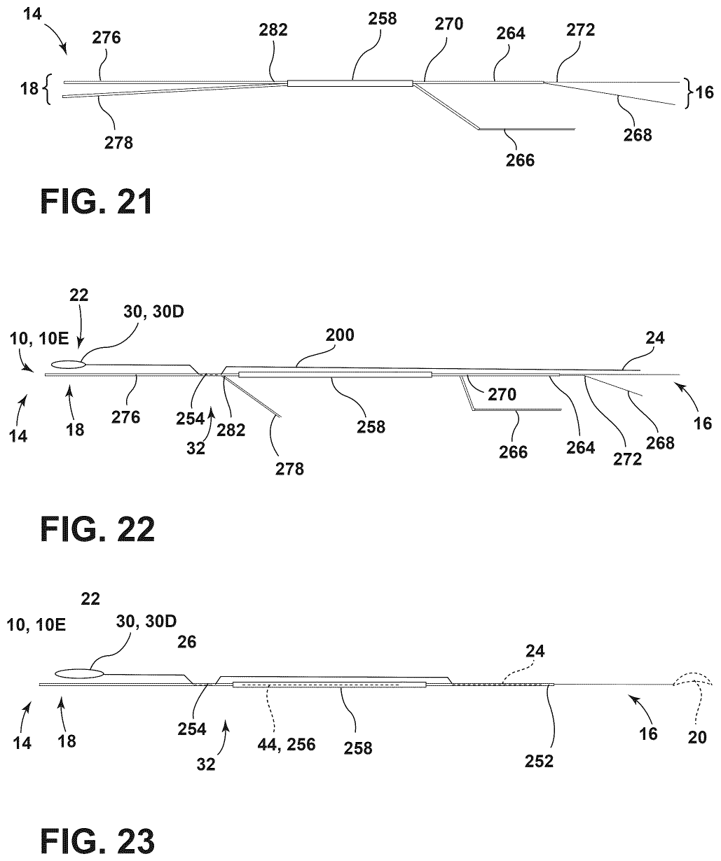

Referring now to FIGS. 21-23, the suture assembly 10E is illustrated, which may be similar to the suture assembly 10D having multiple spliced segments 252, 254 between the repair suture 14 and the shuttle suture 22. The suture assembly 10E may include two spliced segments 252, 254 between the repair suture 14 and the shuttle suture 22 and may omit the third spliced segment 256. Additionally, at least one end 26 of the shuttle suture 22 may not be included in the spliced segments 252, 254. In this illustrated example, the non-looped end 24 may be included in the spliced segment 252 while the shuttle loop 30D may not be included in a spliced segment 254 (e.g., the shuttle loop 30D may be exposed and spaced from the spliced segment 254). The repair suture 14 may be substantially similar as in the suture assembly 10D or may have different aspects to form and locate the spliced segments 252, 254.

Values or measurements are included herein and are understood to be merely exemplary to illustrate the relationship between various sections of the sutures 14, 22 and are, therefore, not limiting. In various non-limiting aspects, the repair suture 14 may be provided and may be between about 30 cm and about 40 cm, such as, for example, about 36 cm. On a first side of the mid-region 258 of the repair suture 14 that includes the first end 16, the repair suture 14 may include three limbs 264, 266, 268. At least one of the limbs 264, 266 may be about one-half the overall length of the repair suture 14, such as about 18 cm. The second limb 266 may be the same length as the first limb 264 or may be shorter.

The repair suture 14 may include a transition section 270 where the two limbs 264, 266 merge into a single limb, and the single limb section may be referred to as the mid-region 258 of the repair suture 14. The mid-region 258 may be at a centerline of the repair suture 14 or may be closer to one of the ends 16, 18 without departing from the teachings herein. The transition section 270 may be about 1 cm in length, which may be included in the length of the one or both limbs 264, 266.

The first limb 264 may further form or extend into an additional third limb 268 at an additional transition section 272. Accordingly, on the first side of the mid-region 258 forming the first end 16, the repair suture 14 may include three limbs 264, 266, 268 with the third limb 268 formed from or coupled with one of the other limbs 264 and having a shorter length. The transition section 272 may be between about 0.2 cm to about 0.5 cm and may extend along the length of the repair suture 14, which may form a portion of the length of the first and third limbs 264, 268. The third limb 268 may be about 10 cm in length.

Referring still to FIGS. 21-24, on an opposing side of the mid-region 258 compared to the first end 16 (e.g., the side including the second end 18), the repair suture 14 may form or include two limbs 276, 278. The limbs 276, 278 may be between about one-half and one-quarter of the length of the repair suture 14. For example, the limbs 276, 278 may be about one-third of the overall length of the repair suture 14, such as about 12 cm. The two limbs 276, 278 on the second side of the mid-region 258 may be shorter than the two longer limbs 264, 266 on the first side of the mid-region 258. On the second side of the mid-region 258 forming the second end 18 of the repair suture 14, the repair suture 14 may have a third transition section 282 where the repair suture 14 can merge from the two limbs 276, 278 and into the single limb mid-region 258. The transition section 282 may be between about 2 mm and about 6 mm along the length of the repair suture 14, which may be included in the length of the two limbs 276, 278.

The transition sections 270, 272, 282 may be spliced sections of the repair suture 14 where two limbs 264, 266, 268, 276, 278 merge or combine into a single limb, respectively. The multi-limb 264, 266, 268, 276, 278 sections of the repair suture 14 may be formed as bifurcated segments. In such examples, during the manufacturing process of the repair suture 14, the strands may be carried in pattern(s) to: form the two limbs 276, 278, be spliced together at the transition section 282 to form the single limb mid-region 258, again be bifurcated to form two limbs 276, 278 on the opposing side of the mid-region 258 and at the transition section 270, and the first limb 264 may be further bifurcated to form the third limb 268 at the transition section 272. The bifurcated limbs 264, 266, 268, 276, 278 of the repair suture 14 may have a lesser width than the single limb mid-region 258, which may reduce the overall size of the suture assembly 10 when the repair suture 14 is combined with the shuttle suture 22.

Alternatively, the different limbs 264, 266, 268, 276, 278 may be manufactured separately. The limbs 264, 266, 268, 276, 278 may then be spliced together at the transition sections 270, 272, 282, respectively, to combine into single limb segments. The repair suture 14 may also be manufactured through a combination of bifurcated segments and limb segments that are spliced together.

Referring still to FIGS. 21-23, the transition sections 270, 272, 282 may provide locations for forming the spliced segments 252, 254 between the repair suture 14 and the shuttle suture 22. The shuttle suture 22 may be arranged proximate to the repair suture 14 to be combined or spliced into the repair suture 14 at one or more locations. Generally, the shuttle end loop 30D may be disposed proximate to the static end 18, and the non-looped end 24 may be disposed proximate to the lead end 16 with the three limbs 264, 266, 268.

The non-looped end 24 of the shuttle suture 22 may be spliced with the lead end 16 of the repair suture 14. The first and second transition sections 270, 272 may form the boundaries for the first spliced segment 252 between the repair suture 14 and the shuttle suture 22. In various aspects, the first end 24 of the shuttle suture 22 may be spliced into the repair suture 14 between the transition sections 270, 272. The shuttle suture 22 may be arranged at or adjacent to the proximal transition section 270 and/or at or adjacent to the distal transition section 272. Additionally or alternatively, the shuttle suture 22 may be spaced from one or both of the transition sections 270, 272.

The spliced segment 252 may be between about 4 cm and about 5 cm in length. The spliced segment 252 may be spaced between about 1 cm and about 2 cm from the single limb mid-region 258. This spacing may align with an end of the first transition section 270. The shuttle suture 22 may be spliced into the first limb 264 at the end of the first transition section 270 and extend toward the second transition section 272. The end 24 of the shuttle suture 22 may be substantially or fully encapsulated in the repair suture 14, such that the end 24 of the shuttle suture 22 may be part of the spliced segment 252. In this way, the repair suture 14 may extend beyond the first end 24 of the shuttle suture 22.

Additionally, the shuttle suture 22 may be spliced into repair suture 14 proximate to the third transition section 282 to form the second spliced segment 254. In the illustrated example, the shuttle suture 22 may be spliced into the first limb 276 in a manner that minimizes the distance between the spliced segment 254 and the end of the spliced limb 276 (e.g., close to where the limbs 276, 278 are spliced or combined at the transition section 282). In non-limiting aspects, the maximum distance between the spliced segment 254 and the merge location of the two limbs 276, 278 may be about 1 mm. The spliced segment 254 may be between about 5 mm and about 10 mm along the length of the sutures 14, 22. The shuttle suture 22 may be interlaced with the repair suture 14 and/or strands thereof to form the combined spliced segments 252, 254 between the sutures 14, 22.

Referring still to FIGS. 20-22, the mid-region 258 of the repair suture 14 may be disposed between the first and third spliced segments 252, 256 formed between the repair suture 14 and the shuttle suture 22. The mid-region 258 may have an increased or greater thickness compared to the ends 16, 18 of the repair suture 14 from being formed as a single limb and may also have a further increased thickness to form the locking feature 32 for the knotless stitch configuration. The repair suture 14 may include the stuffing 44, which may also be referred to as the stuffing suture 44. The stuffing 44 may be additional suture material stuffed within or interlaced with the repair suture 14 to increase the width or thickness and assist in locking the repair suture 14 in the stitch configuration. The stuffing suture 44 may be spliced into the repair suture 14 at the mid-region 258, such as by being inserted into, woven, braided, or otherwise interlaced with the repair suture 14 during the manufacturing process.

The stuffing suture 44 may be spliced into the repair suture 14 at the additional spliced region 262 between the spliced segments 252, 256 with the shuttle suture 22. The thickened portion formed by the stuffing suture 44 may abut or be spaced from one or both of the spliced segments 252, 256. In the illustrated configuration, the stuffing suture 44 may be spaced from both spliced segments 252, 256 and may be closer to the third spliced segment 256 than the first segment 252. The stuffing suture 44 may form the spliced region 262 at the mid-region 258 that extends between about 3 cm and about 5 cm along the length of the repair suture 14.

The stuffing suture 44 may be substantially or fully encapsulated in the repair suture 14 to increase the thickness of the repair suture 14 and reduce any excess material extending from the repair suture 14. The stuffing suture 44 may be arranged off-center in the single limb mid-region 258. As illustrated, the stuffing suture 44 may be closer to a second end of the mid-region 258 (proximate to the third spliced segment 256 on the side with the loop 30D) than a first end (on a side with the non-looped end 24). This may be advantageous for bunching the stuffing 44 to lock the repair suture 14 during the shuttling or tensioning processes while allowing some movement of the stuffing suture 44 within the mid-region 258 and at least substantially containing the stuffing 44 within the mid-region 258. In this configuration, with the third spliced segment 256 (see FIGS. 18-20) being omitted, the second spliced segment 254 may operate as the locking feature 32.

Referring again to FIGS. 21-23, the three limbs 264, 266, 268 at the first end may be combined or joined to form the first end 16 of the repair suture 14, and the two limbs 276, 278 may be combined or joined to form the second end 18 of the repair suture 14. Combining the limbs 264, 266, 268, 276, 278 may result in the repair suture 14 being a single limb along the length thereof. The limbs 264, 266, 268, 276, 278 may be combined via a variety of processes, such as interlacing, weaving, braiding, adhesives, etc. One or both of the ends 16, 18 of the repair suture 14 may be tapered, having a reduced or lesser width/thickness. The tapering may occur during the manufacturing process.

For example, in aspects where the repair suture 14 is formed of interlacing strands, fewer strands of material may be used to form the tapered ends 16, 18. The tapering may also be accomplished by tapering the ends of each limb 264, 266, 268, 276, 278 to collectively form tapered ends 16, 18 of the repair suture 14. The tapered ends 16, 18 may be about 1 cm in length and may assist with reducing friction/resistance during insertion of the suture assembly 10 into/through the tissue T and during the shuttling process. It is contemplated that the lead end 16 of the repair suture 14 may be tapered while the static end 18 may not be as the lead end 16 is inserted through the tissue T. In examples where both the first and second ends 16, 18 are movable or lead ends, both ends 16, 18 may be tapered.

As illustrated in FIGS. 22 and 23, the second spliced segment 254 may be a discrete segment spaced from the ends 18, 26 of the sutures 14, 22. Accordingly, one end of the suture assembly 10E may be a combined end, which may be used for carrying the suture assembly 10E through the tissue T and/or the implant. The non-combined end including the shuttle loop 30D may not be actively inserted through the tissue T prior to the shuttling process. This configuration may leave the shuttle loop 30D as a loose or tail end separated from the repair suture 14.

In certain aspects, the shuttle end loop 30D in the suture assembly 10E may be spliced into and at least partially encapsulated in the repair suture 14, such as in the configuration illustrated in FIGS. 18-20. In such aspects, both ends 24, 26 of the shuttle suture 22 may be spliced into the repair suture 14. This may provide a suture assembly 10 with two combined ends that may be passed through tissue T and/or a valve V in the combined state and then separated for the shuttling process. This may be advantageous for maximizing the efficiency of the insertion process and minimizing the number of separate sutures 14, 22 passing through the tissue T. Moreover, the combined or spliced state of the suture assembly 10 may result in less friction or resistance in passing the suture assembly 10 through the tissue T.

The suture assembly 10E may be coupled with one or more needles 20, 28 for passing the suture assembly 10 through the tissue T and/or the valve V. In examples where the non-looped end 24 is spliced into the repair suture 14 while the looped end 26 is not, a single needle 20 may be coupled to the one or both of the second or lead ends 16, 24 (e.g., a combined lead end of the suture assembly 10E) to pass the suture assembly 10 through the tissue T. In this way, the lead end of the suture assembly 10E coupled with the needle 20 may be passed through the tissue T at multiple locations to position the opposing ends 18, 26 on a same side of the tissue T without passing the ends 18, 26 through the tissue T. Where the end 24 of the shuttle suture 22 is encapsulated in the repair suture 14 such that repair suture 14 extends beyond the end 24 of the shuttle suture 22, at least the lead end 16 of the repair suture 14 may be coupled to the needle 20 and used to insert or carry the suture assembly 10 through the tissue T.

In examples where both ends 24, 26 of the shuttle suture 22 are spliced into the repair suture 14, at least one of the first ends 16, 24 and at least one of the second ends 18, 26 of the suture assembly 10 may be coupled with a respective needle 20, 28, similar to the configuration illustrated in FIGS. 18-20. In such examples, one or both ends of the suture assembly 10 may be passed through the tissue T using the respective needle 20, 28. In additional non-limiting examples, the suture assembly 10 may not include or be coupled with the needle(s) 20, 28. In such examples, the suture assembly 10 may be carried or inserted through tissue T using a device, such as a suture passer or implant device as described further herein.

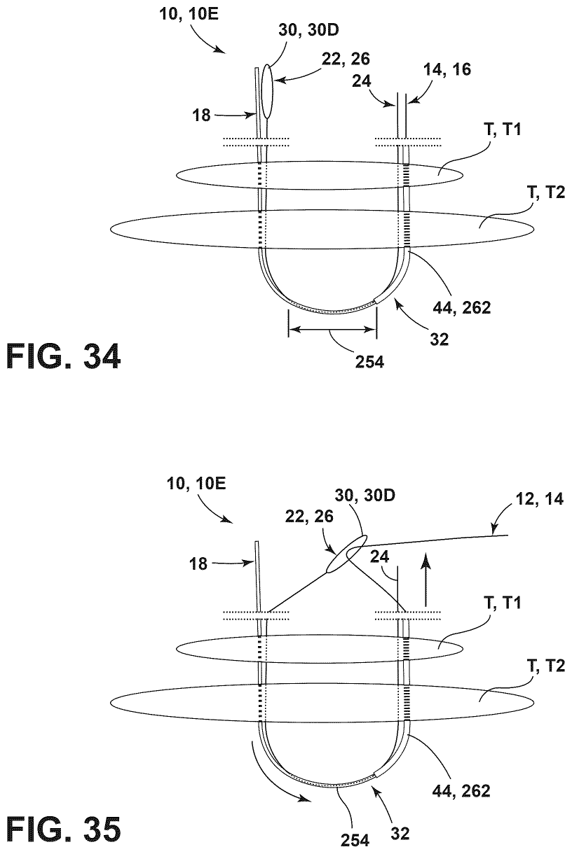

Referring still to FIGS. 21-23, the suture assembly 10E may not include the pledget 12. This may be advantageous for orthopedic procedures, such as attaching two tissue bodies T1, T2 as described herein with respect to FIGS. 34-37. However, the suture assembly 10E may include the pledget 12 without departing from the teachings herein, such as when the suture assembly 10E is used for valve replacement and other implant attachment procedures. In such configurations, the spliced segment 254 between the repair suture 14 and the shuttle suture 22 forming the locking feature 32 may be arranged in or adjacent to the pledget 12 to couple to the suture assembly 10E with the pledget 12, similar to the configurations illustrated in FIGS. 4-8, 10-12, 15, 16, and 20. The pledget 12 may assist with distributing pressure applied by the repair suture 14. The suture assembly 10 may exit the pledget 12 at different locations to create a buttress point for anchoring in tissue T.

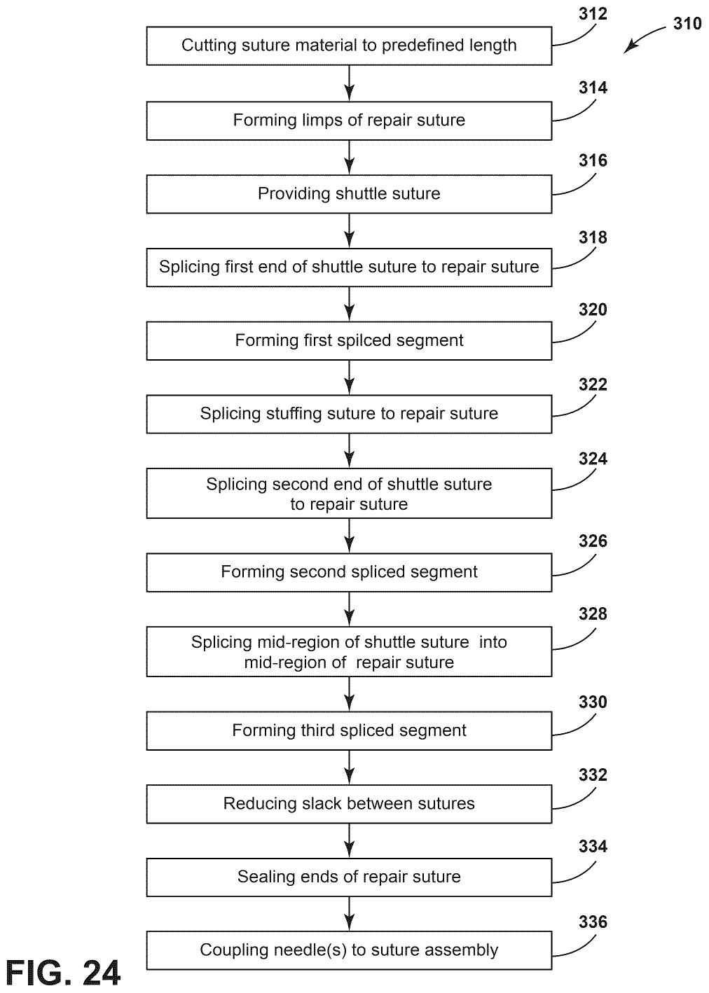

Referring to FIG. 24, as well as FIGS. 18-23, the suture assembly 10 may be manufactured to combine the repair suture 14 with the shuttle suture 22 at one or more of the spliced segments 252, 254, 256 and the stuffing suture 44 at the spliced region 262. A method (310) of manufacturing the suture assembly 10 may include trimming or cutting strands of suture material to the predefined length(s) (312). The repair suture 14 may be formed (314). The repair suture 14 may be formed as a single limb or with multiple limbs 264, 266, 268, 276, 278 (314). The repair suture 14 may be formed during a single process where the strands can be arranged in bifurcated segments and spliced together to form the repair suture 14 with the multiple limbs 264, 266, 268, 276, 278 on each end 16, 18, respectively.

Alternatively, different limbs or segments of suture material may be formed initially, and the segments may be spliced together to form the transition sections 270, 272, 282 and, ultimately, form the repair suture 14 with the mid-region 258 and multiple limbs 264, 266, 268, 276, 278. The suture material may be spliced together to form alternative limbs 264, 266, 268, 276, 278 at specified lengths. The limbs 264, 266, 268, 276, 278 may be trimmed to reduce or prevent fraying and the ends thereof. Trimming the limbs 264, 266, 268, 276, 278 may also ensure that the shuttle suture 22 is substantially or fully encapsulated.

The shuttle suture 22 may be provided (316). The shuttle suture 22 may be spliced into the repair suture 14 (318) and form the first spliced segment 252 (320). The first spliced segment 252 may include the first end 24 of the shuttle suture 22 and may include or be adjacent to the first end 16 of the repair suture 14. The first spliced segment 252 may be formed to be adjacent to the end of the spliced limb 264 proximate to the transition section 270. In certain aspects, the shuttle suture 22 may be spliced into the first limb 264 on the first side of the mid-region 258, which may be the limb 264 that includes the second transition section 272 to form the third limb 268. This spliced segment 254 may be between the two transition sections 272, 282 on the first side of the mid-region 258.

The stuffing 44 or stuffing suture 44 may be spliced into the repair suture 14 at the mid-region 258 (322). The stuffing suture 44 may be centered or off-center within the mid-region 258. The spliced region 262 formed between the repair suture 14 and the stuffing 44 and the stuffing 44 may extend along the length of the mid-region 258, such as between the first transition section 270 and the third transition section 282. The spliced region 262 may be formed between a centerline of the sutures 14, 22 and the first ends 16, 24. The stuffing 44 may be disposed adjacent to the spliced segment 254, 256 between the shuttle suture 22 and the repair suture 14 that forms the locking feature 32 depending on the configuration of the suture assembly 10D, 10E.

The shuttle suture 22 at or proximate to the looped end 26 may be spliced into the repair suture 14 (324). The second spliced segment 254 between the repair suture 14 and the shuttle suture 22 may be formed (326). The shuttle loop 30D may be included in the spliced segment 254. The second end 26, including the end shuttle loop 30D, of the shuttle suture 22 may be substantially or fully encapsulated in the repair suture 14. The second end 18 of the repair suture 14 may extend beyond the second end 26 of the shuttle suture 22. Alternatively, the shuttle loop 30D may remain a loose tail end, and the spliced segment 254 may be spaced from the shuttle loop 30D. In the suture assembly 10E, the second spliced segment 254 may form the locking feature 32.

In certain aspects, such as for the suture assembly 10D, the mid-section 200 of the shuttle suture 22 may be spliced into the mid-region 258 of the repair suture 14 (328). In such aspects, the third spliced segment 256 may be formed between the shuttle suture 22 and the repair suture 14 (330). The shuttle suture 22 and the repair suture 14 may be spliced together at mid-section 200 and the mid-region 258 to form the locking feature 32. The third spliced segment 256 may be formed proximate to the stuffing 44 or stuffing suture 44. The steps 328, 330 may be omitted for the suture assembly 10E with the two spliced segments 252, 254 with the shuttle suture 22. However, any number of spliced segments may be utilized without departing from the teachings herein. Further, while the locking feature 32 is described as the spliced segments 254, 256 to pull the repair suture 14 through itself, the grommet 80 (FIG. 10) may be used without departing from the teachings herein.

Excess length or “slack” of the shuttle suture 22 may be reduced or removed by applying a pulling force to the shuttle suture 22 (332). In configurations with the free looped end 26, the pulling force may be applied to the looped end 26. The pulling force may allow the shuttle suture 22 to move relative to and/or through the repair suture 14 at the spliced segments 252, 254, 256.

The ends 16, 18 of the repair suture 14 may be sealed or formed (334). The limbs 264, 266, 268, 276, 278 at each end 16, 18 may be joined or combined together, such as via adhesive(s). The suture assembly 10 may be completed with the shuttle suture 22 spliced into the repair suture 14 at two segments 252, 254 or three segments 252, 254, 256 and the stuffing suture 44 spliced into the repair suture 14 at the spliced region 262. One or both needles 20, 28 may be coupled with the suture assembly 10 (336). The steps 312-336 of the method 310 may be modified or conducted in different orders, with steps omitted, repeated, performed concurrently, etc. without departing from the teachings herein. For example, the spliced segments 252, 254, 256 and spliced region 262 may be formed in any order without departing from the teachings herein.

The pre-combined suture assembly 10, including the exemplary suture assemblies 10D, 10E, may be advantageous for stitching or suturing multiple tissue bodies T1, T2 together and for implant attachment procedures. The pre-combined suture assembly 10 may maximize the efficiency of the procedure and reduce strands of sutures 14, 22 that are initially inserted through the tissue T, which may reduce resistance in the insertion process. The pre-combined suture assembly 10 may be utilized with two needles 20, 28 for inserting both ends of the suture assembly 10 through the tissue T and/or the implant, similar to the process described in FIGS. 4-8. Two needles 20, 28 may be advantageous when the pledget 12 is utilized to position the pledget 12 against the underside of the tissue T. Additionally or alternatively, the pre-combined suture assembly 10 may be utilized one needle 20. The single needle 20 configuration may be advantageous for inserting a single end of the suture assembly 10 through tissue T, which may be advantageous when the suture assembly 10 omits the pledget 12.

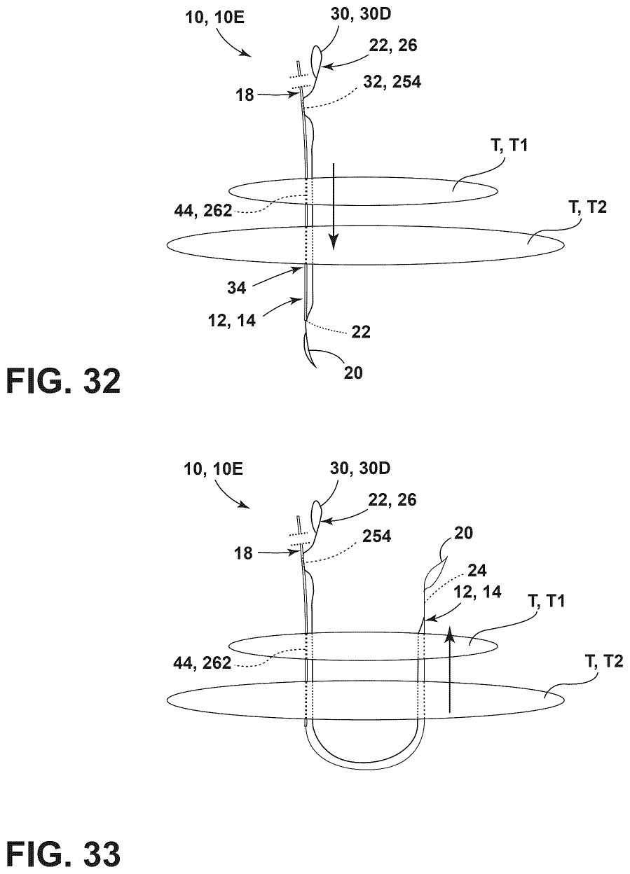

Referring to FIGS. 25-37, when suturing tissue bodies T1, T2, the tissue bodies T1, T2 may be two separate tissues or may be portions of the same tissue T on opposing sides of an incision, cut, or other spacing. Moreover, the tissue bodies T1, T2 may be side-by-side to one another (see FIGS. 25-31) or vertically overlap one another (see FIGS. 32-37). By way of example, the suture assembly 10E is illustrated in a process of connecting two tissue bodies T1, T2.

Referring to FIGS. 25-31, the suture assembly 10 may be utilized to attach or connect two laterally adjacent tissue bodies T1, T2. The tissue bodies T1, T2 may be arranged side-by-side with the incision or cut therebetween. The lead end 16 of the suture assembly 10, which may be or include the lead end 16 of the repair suture 14, may be inserted through the first tissue body T1 in a first or outside-to-inside direction. The lead end 16 may be tapered and may carry the first end 24 of the shuttle suture 22 encapsulated therein. The ends 16, 24 may be carried via the needle 20 or the insertion device. The encapsulated end 24 of the shuttle suture 22 may be advantageous for carrying two sutures 14, 22 through the tissue T at a single time and as a single limb. Further, the encapsulated end 24 may reduce friction or resistance as the suture assembly 10 is carried through the tissue T. For example, the lead end 16 of the repair suture 14 may be thinner or smaller than where the end 24 of the shuttle suture 22 is encapsulated, which may result in a thinner or smaller portion of the suture assembly 10 initially being inserted through the tissue T to prepare for the thicker portion of the suture assembly 10.

Referring to FIGS. 25 and 26, the lead end 16 of the suture assembly 10 may be carried or inserted through the second tissue body T2. Generally, the suture assembly 10 may be moved under the tissue T and across the cut or incision between the tissue bodies T1, T2. The suture assembly 10 may be inserted in a second opposing or inside-to-outside direction through the second tissue body T2 for the ends 16, 24 of the repair suture 14 and the shuttle suture 22 to be arranged on an outer side of the tissue T.

When utilizing the suture assembly 10 for the tissue bodies T1, T2, the suture assembly 10 is inserted or carried through the tissue bodies T1, T2 in opposing directions (e.g., in the first direction and also the second direction). The configuration of inserting a single end of the suture assemblies 10 through the two tissue bodies T1, T2 may be advantageous for minimizing the size of the suture assembly 10 that is carried through the tissue T. For example, using the suture assembly 10E for illustrative purposes, the end of the suture assembly 10 with the shuttle end loop 30D may be larger or thicker, which may be more difficult to carry through the tissue T or result in more resistance/friction. However, each end of the suture assembly 10 may be carried from an inner side to an outer side without departing from the teachings herein, such as when both ends of the suture assembly 10 are coupled with needles 20, 28.

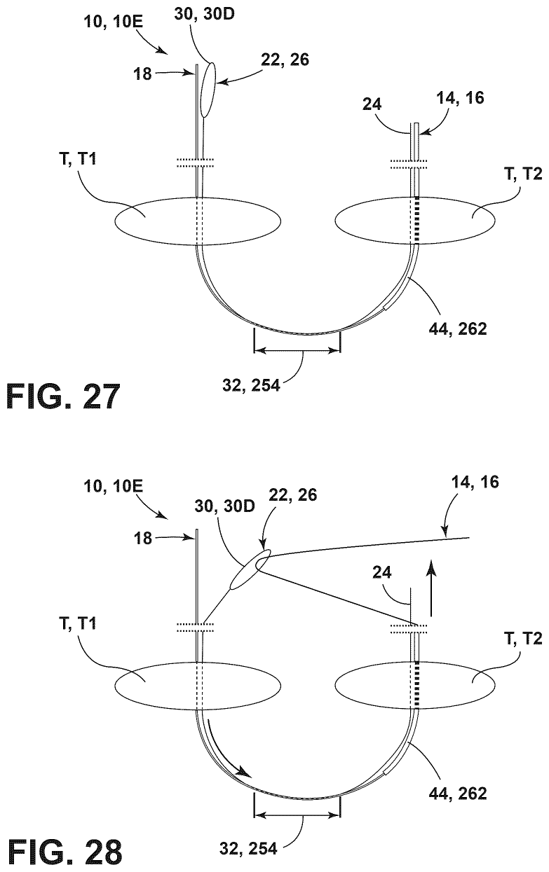

Referring now to FIGS. 27 and 28, the suture assembly 10 may continue to be pulled through the tissue bodies T1, T2 by applying the pulling force or tension to the lead ends 16, 24 of the sutures 14, 22. The pulling force may be applied until the locking feature 32 and the thickened portion formed by the stuffing suture 44 (e.g., the spliced region 262 and/or the mid-region 258) are disposed between the tissue bodies T1, T2. The locking feature 32 may be centrally located between the tissue bodies T1, T2. The spliced region 262 with the stuffing suture 44 may be arranged below the tissue T and between the two tissue bodies T1, T2. As illustrated, the spliced region 262 may be closer to one of the tissue bodies T2. Accordingly, the spliced region 262 may be generally arranged at the cut or incision prior to the shuttling process.

The lead end 16 of the repair suture 14 may be separated from the end 24 of the shuttle suture 22. The suture assembly 10 may be cut to remove the needle 20, which may also separate the sutures 14, 22. The end 24 of the shuttle suture 22 may be moved from within the spliced segment 254 to be a loose tail end 24. Alternatively, one or more of the limbs 264, 266, 268 may be separated from the combined end 16 of the repair suture 14 to provide access to the shuttle suture 22. The lead end 16 of the repair suture 14 may be independently movable relative to the end 24 of the shuttle suture 22.

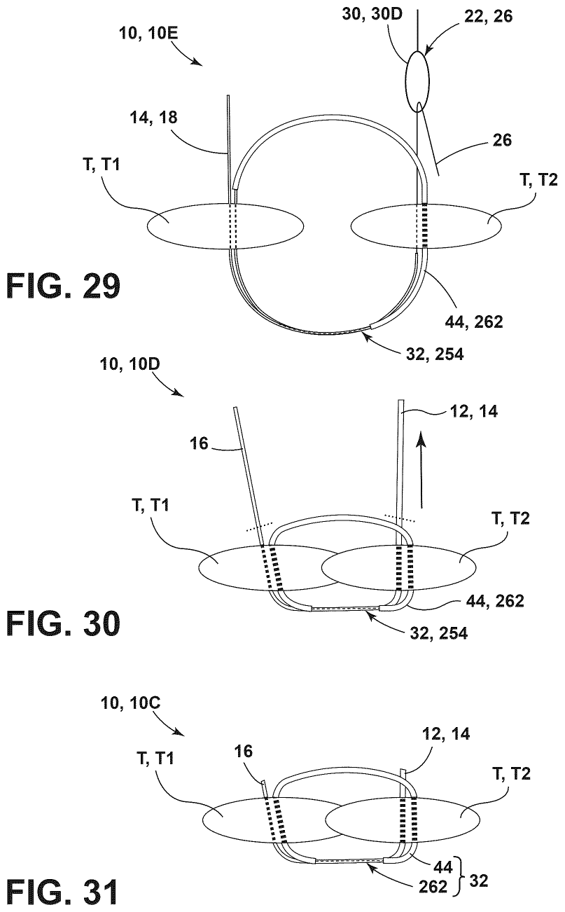

Referring now to FIGS. 28 and 29, the lead or moving end 16 of the repair suture 14 may be threaded through the shuttle end loop 30D, extending across the cut or incision. The first end 24 of the shuttle suture 22 may remain proximate to the second tissue body T2. The repair suture 14 may include one or more fold indicator(s) 56, which may provide visual feedback on where to fold the suture 14. The lead end 16 of the repair suture 14 may be inserted through the shuttle end loop 30D and folded back at the fold indicator 56. The repair suture 14 may generally fold back on itself to extend along itself to form the folded end 16 around the shuttle end loop 30D. This folded arrangement around the shuttle end loop 30D may allow the repair suture 14 to remain engaged with the shuttle end loop 30D as the pulling force is applied to the tensioning end 24 of the shuttle suture 22. The lead end 16 of the repair suture 14 may be tapered, which may be advantageous for forming a smaller folded end 16 for guiding the folded end 16 through the tissue T and the locking feature 32.

The pulling force can be applied to the tensioning end 24 of the shuttle suture 22, which may draw the shuttle end loop 30D and the engaged folded end 16 of the repair suture 14 toward and through the first tissue body T1. A holding force may be applied to the opposing static end 18 of the repair suture 14 to move a portion of the repair suture 14 during the shuttling process while retaining engagement between the repair suture 14 and the tissue bodies T1, T2. During the shuttling process, the repair suture 14 may be looped over the spacing between the tissue bodies T1, T2 and drawn through the first tissue body T1 from the outer side to the inner side and toward the locking feature 32.

The locking feature 32, which may be the second spliced segment 254, may be the location where the shuttle suture 22 is interlaced, woven, or otherwise extended through the repair suture 14. The pulling force at the tensioning end 24 of the shuttle suture 22 may be configured to draw the shuttle suture 22 and the engaged folded end 16 through the spliced segment 254. With the tension or pulling force being applied to the end 24 of the shuttle suture 22, the shuttle suture 22 may be drawn through the spliced segment 254 or other locking feature 32. As the shuttle end loop 30D is drawn through spliced segment 254, the folded end 16 of the repair suture 14 may also be drawn through the spliced segment 254 or locking feature 32. This movement may pull the shuttle suture 22 out of the repair suture 14 and the tissue T and the repair suture 14 through itself. The tapered nature of the end 16 of the repair suture 14 may be advantageous for pulling the repair suture 14 through itself with less resistance.

The repair suture 14 may include the thickened portion with the stuffing suture 44 proximate to the locking feature 32 where the repair suture 14 is pulled through itself. The repair suture 14 being drawn through the spliced segment 254 and tensioning of the suture 14 may cause the stuffing suture 44 to compress, wrinkle, or bunch within the mid-region 258 of the repair suture 14. The bunching of the thickened portion may form a locking trap (e.g., the locking feature 32) that assists with locking the repair suture 14 in the looped or stitch configuration. The resistance caused by the thicker stuffed portion may lock the repair suture 14 under tension. The locking trap may allow re-tensioning or further tightening while reducing or preventing loosening of the repair suture 14.

Referring now to FIGS. 28-31, the pulling force may continue to draw the shuttle end loop 30D and the folded end 16 through the second tissue body T2 from the inner side to the outer side, forming a full loop with the repair suture 14 across and between the tissue bodies T1, T2. The shuttle suture 22 may be fully removed from the tissue T upon completion of the shuttling process. The pulling force can be applied to the lead end 16 of the repair suture 14 to tension or tighten the repair suture 14, drawing or compressing the tissue bodies T1, T2 together. The pulling force can be applied until the tissue bodies T1, T2 abut or overlap one another, closing the incision between the tissue bodies T1, T2. In other words, the pulling force can be applied until sufficient compression occurs between the tissue bodies T1, T2. The knotless locking feature 32 may retain the repair suture 14 under tension and, consequently, the compression between the tissue bodies T1, T2. The ends 16, 18 of the repair suture 14 may then be cut proximate to the tissue T to reduce excess suture 14 length in the patient anatomy.

Referring to FIGS. 32-37, similarly, the suture assembly 10 may also be used for tissue bodies T1, T2 that are stacked over one another. In such examples, the tissue bodies T1, T2 may be vertically stacked or overlapped with the cut, incision, or spacing separating two layers of tissue bodies T1, T2. The lead end of the suture assembly 10, including the lead ends 16, 24 of the sutures 14, 22, may be inserted through the first outer tissue body T1, across the spacing, and through the second inner tissue body T2. Generally, the two inward insertion locations (illustrated on the left side in FIGS. 32 and 33) may be aligned with one another to allow movement of the tissue bodies T1, T2 toward one another while reducing side-to-side movement between the tissue bodies T1, T2, which may increase stress on the tissue T.

Referring still to FIGS. 32-35, the suture assembly 10 may extend along an underside of the inner tissue body T2. The lead end 16 may be inserted or carried through the inner tissue body T2, across the spacing, and through the outer tissue body T1. The two outward through locations (illustrated on the right side in FIGS. 32 and 33) may generally be parallel with one another to provide a compression between the tissue bodies T1, T2 with reduced side-to-side movement. The suture assembly 10 may extend parallel to itself as it extends through the tissue bodies T1, T2, which may reduce bunching or lateral movement of the tissue bodies T1, T2.

The pulling force may be applied to the lead end of the suture assembly 10 until the locking feature 32 and the spliced region 262 with the stuffing suture 44 are disposed below the inner tissue body T2. The thickened portion may be advantageous under the tissue T for distributing the force applied by the repair suture 14 on the tissue T. The ends 16, 18, 24, 26 of the shuttle suture 22 and the repair suture 14 may be adjusted to allow independent movement of the repair suture 14 relative to the shuttle suture 22. For example, the needle 20 may be cut from the suture assembly 10, allowing separation of the sutures 14, 22.

Referring to FIGS. 35-37, the shuttle suture 22 may be utilized for the shuttling process. The lead end 16 of the repair suture 14 may be moved across the outer tissue body T2, inserted through the shuttle end loop 30D, and folded back on itself. The pulling force may be applied to the tensioning end 24 of the shuttle suture 22 to pull the shuttle end loop 30D and, consequently, the folded end 16 of the repair suture 14. The folded end 16 may be drawn by the shuttle suture 22 in a path including: through the outer tissue body T1, across the spacing, through the inner tissue body T2, along the underside of the inner tissue body T1 and through the locking feature 32/spliced segment 254, through the inner tissue body T2, across the spacing, and through the outer tissue body T1. This path may form the looped or stitch configuration to compress the tissue bodies T1, T2 together and remove the shuttle suture 22 from the tissue T.

The repair suture 14 may be drawn through itself at the spliced segment 254 and cause the stuffing suture 44 to bunch, forming the locking trap for locking the repair suture 14 under tension in the stitch configuration. The pulling force may be applied to the lead end 16 of the repair suture 14 to compress the tissue bodies T1, T2 together. The knotless locking feature 32 may retain the repair suture 14 under tension and the compression between the tissue bodies T1, T2. Upon sufficient compression between the tissue bodies T1, T2, the ends 16, 18 of the repair suture 14 may be trimmed or cut proximate to the tissue T.

The suture assembly 10 may be a knotless orthopedic suture assembly 10 for connecting multiple tissue bodies T1, T2, which may be separate tissues T or portions of the same tissue T. The suture assembly 10 may utilize the knotless locking feature 32, which can hold the repair suture 14 under tension without the use of more traditional knots and crimps. This may reduce the profile of the suture assembly 10 that remains in the patient anatomy and may reduce implants in the patient. The suture assembly 10 may combine the two sutures 14, 22, allowing for increased efficiency in carrying the sutures 14, 22 through the tissue T and minimizing resistance caused by the suture assembly 10. Though discussed primarily in reference to connecting tissue bodies T1, T2, it shall be understood that knotless suture assembly 10 may be applied in a variety of applications and procedures.

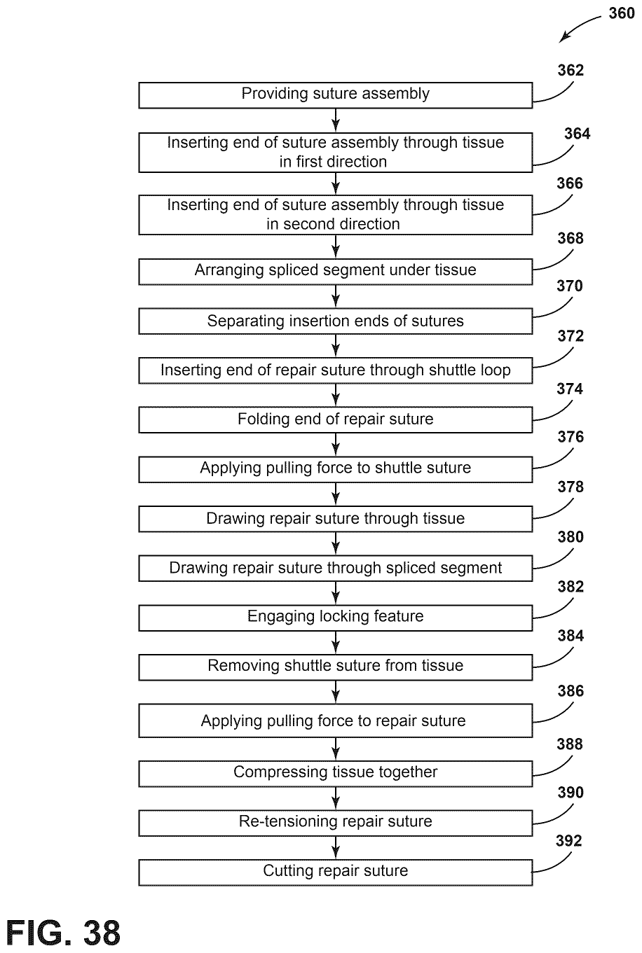

Referring to FIG. 38, as well as FIGS. 25-37, the suture assembly 10 may be utilized for orthopedic procedures and methods (360) to connect tissue bodies T1, T2 at an incision, cut, or other spacing. For suturing multiple tissue bodies T1, T2, the suture assembly 10 can be provided (362), which may be formed using the method of manufacturing (310) described herein with respect to FIG. 24 or another manufacturing method. The suture assembly 10 may be preassembled or preformed with the repair suture 14 and the shuttle suture 22 spliced together. The lead end of the suture assembly 10 may be carried or inserted through the tissue T in the first or outward-to-inward direction (364). Typically, the lead end may be the combined first ends 16, 24 of the sutures 14, 22. The suture assembly 10 may be carried through the first tissue body T1 when the tissue bodies T1, T2 are laterally adjacent to one another or both tissue bodies T1, T2 when the tissue bodies T1, T2 overlap.

In certain aspects, the suture assembly 10 may be inserted through the pledget 12 at the underside of the tissue T. This may allow the suture assembly 10 to be inserted in a single direction via one needle 20 and include the pledget 12. The needle 20 may be used to insert or carry the suture assembly 10 through the pledget 12. The pledget 12 may assist with distributing pressure from the repair suture 14 and may assist in closing the opening between the tissue bodies T1, T2.

The lead end of the suture assembly 10 may be carried or inserted through the tissue T in the second opposing or inward-to-outward direction (366). This may be the second tissue body T2 when the tissue bodies T1, T2 are laterally adjacent to one another or both tissue bodies T1, T2 when the tissue bodies T1, T2 overlap. A pulling force may be applied to the lead end of the suture assembly 10 to position the locking feature 32 and the spliced region 262 with the stuffing suture 44 below the tissue T (368). The insertion or lead ends 16, 24 of the repair suture 14 and the shuttle suture 22 may be separated or otherwise adjusted to allow independent movement of the sutures 14, 22 relative to one another (370). The lead end 16 of the repair suture 14 may be moved across the tissue T and inserted through the shuttle end loop 30D (372). The repair suture 14 may be folded back on itself (374). The pulling force may be applied to the first end 24 of the shuttle suture 22 to move the repair suture 14 along a shuttling path (376).

The folded end 16 of the repair suture 14 may be guided through the tissue T (378) (e.g., one or both of the tissue bodies T1, T2) in the outward-to-inward direction and through the spliced segment 252 (380), engaging the locking feature 32 and causing the thickened portion to bunch and lock the repair suture 14 (382). The folded end 16 of the repair suture 14 may be guided through the tissue T (e.g., one or both of the tissue bodies T1, T2) in the inward-to-outward direction, and the shuttle suture 22 may be withdrawn or removed from the tissue T (384). The pulling force may be applied to the lead end 16 of the repair suture 14 (386) and the tissue bodies T1, T2 can be compressed together (388). During the shuttling and tensioning processes, the second end 18 of the repair suture 14 may remain static. Alternatively, the pulling force may also be applied to the second end 18 to assist with tensioning the repair suture 14 and compressing the tissue bodies T1, T2 together. The repair suture 14 may be re-tensioned (390) and the ends 16, 18 trimmed or cut (392). The steps 362-392 of the method 360 may be modified or conducted in different orders, with steps omitted, repeated, performed concurrently, etc. without departing from the teachings herein.

The suture assembly 10 may be inserted through the tissue T or tissue bodies T1, T2 multiple times prior to the shuttling process without departing from the teachings herein. This may allow the formation of multiple “stitches” formed from the same suture assembly 10, which may be advantageous for providing more connection points between two tissue bodies T1, T2. More connection points may assist with providing more even compression between the tissue bodies T1, T2 and/or providing more connections along a longer incision or opening between tissue bodies T1, T2. Additionally or alternatively, multiple suture pairs 40, each formed from an integrated suture assembly 10, may be utilized along a single incision or cut between tissue bodies T1, T2. The ability to re-tension the repair sutures 14 may be advantageous when utilizing multiple suture pairs 40 to distribute pressure and provide better alignment between the tissue bodies T1, T2.

While described with a single insertion end, both ends of the suture assembly 10 may be inserted through the tissue T. In such examples, the first needle 20 carrying the first ends 16, 24 can be inserted through the first tissue body T1, and the second needle 28 carrying the second ends 18, 26 can be inserted through the second tissue body T2. The locking feature 32 may be arranged below and/or between the tissue bodies T1, T2. When using two needles 20, 26, the pledget 12 may also be used. The needles 20, 28 may be removed from the suture assembly 10 and the shuttling process may be performed to compress the tissue bodies T1, T2 together. This process may be substantially similar to the steps 104-124 of the method 100 described with reference to FIG. 11, as well as the methods 150, 220 in FIGS. 14 and 17, respectively.

The suture assembly 10 may be advantageous for inserting a single combined assembly 10 through the tissue T to then be secured via the knotless configuration. The suture assembly 10 may be advantageous for orthopedic procedures, joining the multiple tissue bodies T1, T2 together and maintaining compression therebetween. The knotless configuration may allow the repair suture 14 to be re-tensioned, continuing to apply sufficient compression between the tissue bodies T1, T2. The suture assembly 10 may also have a smaller overall profile when omitting the pledget 12 and may have a smaller profile for being drawn through the tissue T combining the sutures 14, 22 together. The suture assembly 10 may also be used for valve replacement procedures, where the suture assembly 10 may attach the tissue T with the valve in a similar manner as the two tissue bodies T1, T2 (e.g., in a single direction with one needle 20) or similar to the processes described above with the suture assemblies 10A-10C.

Referring again to FIGS. 1-38, the suture assemblies 10 described herein may be re-tensioned during the valve replacement procedure, implant attachment procedure, and tissue connecting procedures. This may allow for an increased seal, more accurate alignment, such as between the valve V and the tissue T, and/or increased compression between the tissue bodies T1, T2. Using a valve replacement procedure as an example, conventional sutures are generally tensioned a single time without the ability to be re-tensioned, which can result in limitations in adjusting the valve V and, consequently, improper alignment between the valve V and the tissue T. Often, during the valve replacement procedure, the repair sutures 14 are initially provided at the commissures C where the leaflets meet. For an aortic valve replacement, there are generally three commissures C (see FIG. 2). With more conventional sutures, the sutures are generally tightened in sequence at the commissures C and, once tightened, the sutures can be knotted or a crimp is crimped to lock or hold the sutures. Once the suture is knotted or the crimp is crimped, the sutures are generally fixed and may not be re-tensioned or adjusted. If the valve V shifted during the procedure, which can be common based on tension being applied to the conventional sutures in sequences, the valve V is generally fixed in the slightly displaced position.

The re-tensioning that can be accomplished via the suture assemblies 10 disclosed herein may provide for better alignment of the replacement valve V. For example, using the suture assembly 10, the repair sutures 14 may initially be tensioned at the commissures C to attach the replacement valve V to the tissue T. During this process, the replacement valve V may become displaced due to the temporarily uneven tension of the repair sutures 14, which can be more common in mechanical valves V. Each of the suture assemblies 10 can then be re-tensioned multiple times during the valve replacement procedure to assist with balancing the repair or fixation of the replacement valve V and positioning of the replacement valve V. Accordingly, the ability to re-tension the repair suture 14 may allow for better alignment of the valve V, which can also result in a better fluid seal between the valve V and the tissue T. The suture assemblies 10 can also be re-tensioned for other implant procedures and for connecting tissue bodies T1, T2.

Further, in various aspects, the suture assemblies 10 may also be utilized with implant clips. In such configurations, the implant clips may engage the looped repair suture 14 formed from the shuttling process before the compression and tensioning in the stitch configuration. The implant clips may engage adjacent repair sutures 14, which may provide a greater surface area for compression between the tissue bodies T1, T2 and/or between the valve V and the tissue T.

Referring still to FIGS. 1-38, in certain aspects, the suture assembly 10 may also include smaller/thinner sutures 14, 22, which may reduce the footprint of the suture assembly 10. One or both of the sutures 14, 22 may be 0, 2-0, 4-0, or 5-0 sutures depending on the configuration of the suture assembly 10. This sizing may provide a balance between being sufficiently narrow for the sutures 14, 22 to be threaded through various features, including the locking feature 32, and having a sufficient thickness to form the spliced section 70 and/or spliced segments 252, 254, 256 of the repair suture 14. This sizing can also provide sufficient size for the bifurcated section of the shuttle link 30A of the shuttle suture 22, the shuttle receiving area 30B, the folded shuttle loop 30C, and/or the end shuttle loop 30D when used. The sutures 14, 22 may have a variety of configurations, such as tape-like, flat, tubular, tubular that compresses flat, etc. to balance between size and function. The sutures 14, 22 may also be manufactured to promote multiple spliced segments 252, 254, 256 between the sutures 14, 22 to form a combined or integrated assembly 10.

Additionally, different portions of the sutures 14, 22 may have different configurations. For example, the repair suture 14 may have a tape-like portion that may become pledget-like, which can include the spliced section 70, the spliced segments 252, 254, 256, and/or the spliced region 262. The pledget-like section of the repair suture 14 may be coupled with the pledget 12 and/or replace the pledget 12. The pledget-like section may be advantageous in the suture assemblies 10D, 10E to better distribute pressure from the repair suture 14 while providing an integrated assembly 10 formed with a single insertion end.

The suture assemblies 10 can also include the pledget 12, which may also have a variety of configurations, including the flat pledget 12A, the folded pledget 12B, and the annular pledget 12C. For example, the pledget 12 may include a braided jacket to be built into the round suture, similar to a fiber tape. This may further reduce the size of the overall assembly 10 and number of components utilized in the replacement procedure.

Additionally, the sutures 14, 22 may have increased strength compared to conventional sutures. One or both of the repair suture 14 and the shuttle suture 22 may be constructed of polyethylene. The polyethylene construction may be more inert and stronger than a conventional polyester suture. The increased strength may be advantageous for applying the pulling force to the sutures 14, 22 during the shuttling process where conventional polyester sutures may have an increased risk of shredding or breaking down during this process. The use of polyethylene may provide the strength profile for the shuttling process.

Referring still to FIGS. 1-38, one or both of the repair sutures 14 and the shuttle sutures 22 may be manufactured to have different visual indicators or differentiators to guide the use of the suture assembly 10 during the valve replacement procedure. These visual indicators include the cut indicators 50-54, the fold indicator(s) 56, and/or the insertion indicator(s) 132. Additionally, the visual differentiation may assist with distinguishing the shuttle suture 22 from the repair suture 14, as well as the location on the shuttle suture 22 to apply the pulling force. For example, the two sutures 14, 22 may be manufactured in different colors or with different patterns along the length of the sutures 14, 22. Additionally, for the suture assemblies 10A, 10B, the first end 24 of the shuttle suture 22 may have a different pattern than the second end 26, such as black stripes on one side of the shuttle suture 22, which may be advantageous for indicating the pulling location for the shuttle suture 22. The differentiation may also be helpful when separating spliced sutures 14, 22 for the shuttling process.

The visual indicators and differentiators may assist with providing markers for guiding the procedure, such as the cut indicators 50-54, the fold indicator(s) 56, and/or the insertion indicator(s) 132. The differentiation between the two sutures 14, 22 and identifying the pulling location may be advantageous when multiple suture pairs 40 are arranged around a single annulus A or incision between tissue bodies T1, T2. This may assist in properly and more efficiently identifying the different sutures 14, 22, particularly if two sutures 14, 22 from different suture pairs 40 become crossed during the replacement procedure.

Referring still to FIGS. 1-38, the shuttle sutures 22 and the repair sutures 14 may be manufactured to have different widths and different components. The shuttle sutures 22 may be woven as a single limb, bifurcated into two limbs to form the shuttle link 30A, and then again woven as a single limb. This manufacturing process may allow the shuttle link 30A to be formed without significantly increasing the width of the shuttle suture 22 at the shuttle link 30A. Alternatively, the shuttle sutures 22 may be folded to form two limbs that define the folded shuttle loop 30C or the end shuttle loop 30D. Further, the repair suture 14 may include one or more of multiple limbs 264, 266, 268, 276, 278, transition sections, 270, 272, 282, spliced segments 252, 254, 256, and the spliced region 262.

In additional non-limiting examples, both the sutures 14, 22 may have one limb along a substantial portion, or an entirety, of their lengths. When folding the shuttle suture 22 to form the shuttle loops 300, 30D or using the single limb with the shuttle receiving area 30B, the width/thickness of the shuttle suture 22 may be smaller than when using the bifurcated shuttle link 30A. Additionally, during a weaving, braiding, or interlacing process for the repair suture 14, a number of strands in the repair suture 14 may be reduced to form the narrower and thinner first end 16 and/or second end 18. The different visual indicators may also be added or incorporated into the sutures 14, 22 during one or more stages of the manufacturing process.

As described herein, the first and second needles 20, 28 may be utilized to carry portions of the sutures 14, 22 through the tissue T and/or the valve V before being removed from the suture assembly 10. It is contemplated that certain devices may be utilized in lieu of the needles 20, 28 for carrying the sutures 14, 22 through the tissue T and/or the implant without departing from the teachings herein. In such examples, the device may function similarly to the needles 20, 28 for engaging the sutures 14, 22, carrying the sutures 14, 22, and then disengaging from the sutures 14, 22 for the shuttling process. In other words, the needles 20, 28 may not be included in the suture assembly 10, rather the device is utilized with the sutures 14, 22.

For example, a suture passer device may be utilized with the suture assembly 10. The suture passer device may include a distal jaw with at least one movable jaw member and a movable needle. The jaw may be utilized for holding and moving the tissue. The jaw may also support the needle, which can be selectively engaged with the suture 14, 22 to be passed. In certain aspects, a user may load one or both of the sutures 14, 22 onto the needle of the suture passer. Additionally or alternatively, the suture passer may be configured to automatically re-load one or both of the sutures 14, 22 onto the needle. The suture passer may be used for carrying the sutures 14, 22 through the tissue T and/or the valve V without using an attached needle 20, 28. In other words, the needle can remain as part of the suture passer, omitting the process of removing the needles 20, 28 from the suture assembly 10.

In an additional non-limiting example, an implant device can be used with the suture assembly 10 for placing the suture assembly 10 in the tissue T. The implant device may carry or house one or both sutures 14, 22 and/or the pledget 12. The suture assembly 10 may be housed in a sheath of the implant device. A needle tip of the implant device can be inserted through the tissue T and/or valve V and the user can deploy a portion of the suture assembly 10, such one section as the sutures 14, 22. This first section of the sutures 14, 22 may be the portions coupled with the first needle 20 in configurations utilizing the attached needles 20, 28. The user can then adjust the implant device to load a second section of the suture assembly 10 to be implanted. The second section of the sutures 14, 22 may be the portions coupled with the second needle 28 in configurations utilizing the attached needles 20, 28. The implant device can be removed from the tissue T and then re-inserted through the tissue T at a second location to deploy or carry the second section of the sutures 14, 22 through the tissue T at the second location to form the stitch arrangement.

The suture assembly 10 disclosed herein generally includes the attached needles 20, 28 for carrying the sutures 14, 22 through the tissue T and/or the valve V for forming the knotless stitch configuration. The suture assembly 10 may also utilize a device for passing the sutures 14, 22, such as the suture passer device or implant device, rather than one or both of the attached needles 20, 28. The suture assembly 10 may be configured substantially similarly with and without the attached needles 20, 28 for forming the stitch configuration with the shuttling process to provide a knotless stitch that can be tensioned and re-tensioned.

Referring still to FIGS. 1-38, the suture assemblies 10 disclosed herein and use of the assemblies 10 may provide for a variety of advantages. For example, the suture assemblies 10 may include two needles 20, 28 for each suture pair 40, which can allow the pledget 12 to be disposed under the tissue T. Also, the two needles 20, 28 may allow both of the sutures 14, 22 to be directed through the tissue T and the replacement valve V, which can increase engagement and compression between the tissue T and the replacement valve V to provide a better fluid-tight or blood-tight seal for high-pressure blood flow. The suture assemblies 10 may also be used with a single needle 20 for a single end being inserted through the tissue T, which may be advantageous for orthopedic approaches, such as connecting issue bodies T1, T2. The suture assembly 10 may be used with the pledget 12, which may provide pressure distribution and reinforcement for the tissue T during the tensioning and re-tensioning processes. The suture assembly 10 may also be used without the pledget 12 for single-direction insertion or other procedures.

Additionally, one or both of the sutures 14, 22 may be constructed of polyethylene to increase the strength profile of the sutures 14, 22 for the shuttling process compared to more conventional polyester sutures. Further, the shuttle suture 22 may be bifurcated to form the shuttle link 30A, may have the shuttle receiving area 30B, or may be folded to form the folded shuttle loops 30C, 30D. The repair suture 14 may have the narrower end(s) 16, 18 to assist with the sutures 14, 22 being drawn through the locking feature 32, such as through the spliced section 70 or spliced sections 254, 256 of the repair suture 14, with reduced or minimal resistance. Moreover, the suture assemblies 10 disclosed herein may reduce or eliminate metal components, such as more conventional crimps. In this regard, the suture assemblies 10 may also reduce or eliminate components that remain on the outer side of the replacement valve V (opposite the side that engages the tissue T) or the tissue T, where traditional knots and crimps can remain. The suture assemblies 10 may each be a knotless assembly 10, which may be more convenient and efficient when operating in smaller areas or where multiple suture assemblies 10 are utilized.

Additionally, the pre-connected sutures 14, 22 may allow the surgeon to insert a single assembly 10 through the tissue T and secure the tissue bodies T1, T2 or the tissue T and the implant (e.g., the valve V) with a knotless suture configuration. The suture assembly 10 may also have a smaller overall profile when omitting the pledget 12 and may have a smaller profile for being drawn through the tissue T by being integrated or combined. The suture assembly 10 may include one end 24 of the shuttle suture 22 encapsulated in the repair suture 14 or both ends 24, 26 encapsulated in the repair suture 14. Different methods may be used with the suture assembly 10 depending on the configuration, such as one needle 20 with one end being inserted through tissue T, two needles 20, 28 with both ends being inserted through tissue T, or the use of an insertion device. The suture assemblies 10 may be utilized for attaching implants and for orthopedic procedures, such as attaching tissue bodies T1, T2 together. Additional benefits or advantages would be realized and/or achieved.

The device disclosed herein is further summarized in the following paragraphs and is further characterized by combinations of any and all various aspects described herein.

According to an aspect of the present disclosure, a knotless valve suture assembly may include a pledget. A repair suture may have a proximal end fixedly coupled to the pledget and a distal end coupled to a first needle. A shuttle suture may extend through the pledget. The shuttle suture may have a first end coupled to the first needle and a second end coupled to a second needle. The shuttle suture may include a shuttle link proximate to the second needle. A locking feature may be coupled to the pledget. The locking feature may be configured to retain the repair suture under tension when the repair suture is threaded through the locking feature.

According to an aspect of the present disclosure, a shuttle suture may include a bifurcated section that forms a shuttle link.

According to an aspect of the present disclosure, a locking feature may be a spliced section of a repair suture disposed within a pledget, and a shuttle suture may be configured to extend through the spliced section.

According to an aspect of the present disclosure, a locking feature may be a grommet including outer barbs to engage a pledget.

According to an aspect of the present disclosure, a distal end of a repair suture may taper to a lesser width compared to a proximal end.

According to an aspect of the present disclosure, at least one of a shuttle suture and a repair suture may be constructed of polyethylene.

According to an aspect of the present disclosure, a shuttle suture may be visually differentiated from a repair suture by at least one of a color and a pattern.

According to an aspect of the present disclosure, a first end of a shuttle suture may be visually differentiated from a second end of the shuttle suture by at least one of a color and a pattern.

According to an aspect of the present disclosure, a pledget may be folded to form a first layer and a second layer, and a repair suture and a shuttle suture may extend through a middle of the first layer and the second layer.

According to an aspect of the present disclosure, a knotless valve suture assembly may include a pledget defining a ring shape and multiple suture pairs coupled to the pledget. Each suture pair may include a repair suture having a proximal end that may be fixedly coupled to the pledget and a distal end that may be coupled to a first needle, as well as a shuttle suture that may have a first end coupled to the first needle and a second end that may be coupled to a second needle. The shuttle suture may extend through the pledget proximate to the repair suture. The shuttle suture may include a shuttle link proximate to the second needle to receive the distal end of the repair suture.

According to an aspect of the present disclosure, a method of replacing a cardiovascular valve may include one or more of: inserting a first needle through tissue and a replacement valve, wherein the first needle is coupled to a distal end of a repair suture and a first end of a shuttle suture; inserting a second needle through the tissue and the replacement valve to position a pledget against an inner surface of the tissue, wherein the second needle is coupled to a second end of the shuttle suture; removing the first and second needles; inserting the distal end of the repair suture through a shuttle link of the shuttle suture; applying a pulling force to the first end of the shuttle suture to pull the second end of the shuttle suture and the distal end of the repair suture through the pledget and a locking feature-coupled with the pledget; and tensioning and locking the repair suture.

According to an aspect of the present disclosure, a method may include a step of tensioning and locking a repair suture, which may include threading the repair suture through a spliced section of the repair suture to lock the repair suture.

According to an aspect of the present disclosure, a method may include re-tensioning a repair suture.

According to an aspect of the present disclosure, a knotless valve suture assembly may include a pledget, a repair suture, and a shuttle suture. The repair suture may have a proximal end fixedly coupled to the pledget and a distal end coupled to a first needle. The repair suture may include a locking feature. The shuttle suture may have a first end and a second end each coupled to the first needle. The shuttle suture may be folded and may extend through the locking feature to form a shuttle loop. The shuttle loop may be operably coupled to a second needle via a connecting suture. The shuttle loop may be configured to guide the repair suture through the locking feature to retain the repair suture under tension when the repair suture is threaded through the locking feature.

According to an aspect of the present disclosure, a locking feature may be a spliced section of a repair suture.

According to an aspect of the present disclosure, a shuttle suture may be a monofilament suture.

According to an aspect of the present disclosure, a method of replacing a cardiovascular valve may include one or more of: inserting a first needle through tissue and a replacement valve, where the first needle is coupled to a distal end of a repair suture and a first and second ends of a shuttle suture; inserting a second needle through the tissue and the replacement valve to position a pledget against an inner surface of the tissue, where the second needle is coupled to a shuttle loop of the shuttle suture; removing the first and second needles; inserting the distal end of the repair suture through the shuttle loop; applying a pulling force to the first and second ends of the shuttle suture to pull the shuttle loop and the distal end of the repair suture through the pledget and a locking feature; and tensioning and locking the repair suture with the locking feature.

According to an aspect of the present disclosure, a knotless valve suture assembly may include a pledget, a repair suture that may have a proximal end fixedly coupled to the pledget and a distal end coupled to a first needle, a locking feature that may be coupled to the pledget, and a shuttle suture that may have a first end coupled to the first needle and a second end coupled to a second needle. The repair suture may be configured to be inserted through the shuttle suture via the first needle and guided through the locking feature by the shuttle suture to retain the repair suture under tension when the repair suture is threaded through the locking feature.

According to an aspect of the present disclosure, a method of replacing a cardiovascular valve may include one or more of: inserting a first needle through tissue and a replacement valve, where the first needle is coupled to a distal end of a repair suture and a first end of a shuttle suture; inserting a second needle through the tissue and the replacement valve to position a pledget against an inner surface of the tissue, where the second needle is coupled to a second end of the shuttle suture; removing the first needle from the first end of the shuttle suture; inserting the distal end of the repair suture through the shuttle suture using the first needle; removing the first needle from the distal end of the repair suture and the second needle from the second end of the suture shuttle; applying a pulling force to the first end of the shuttle suture to pull the second end of the shuttle suture and the distal end of the repair suture through the pledget and a locking feature; and tensioning and locking the repair suture with the locking feature.

According to an aspect of the present disclosure, a knotless valve suture assembly may include a pledget, first and second needles, a repair suture that may have a proximal end fixedly coupled to the pledget and a distal end coupled to the first needle, and a shuttle suture that may extend through the pledget. The shuttle suture may be operably coupled with the first and second needles. The shuttle suture may include a shuttling feature. A locking feature may be coupled to the pledget. The locking feature may be configured to retain the repair suture under tension when the repair suture is threaded through the locking feature by the shuttle suture.

According to an aspect of the present disclosure, a first end of a shuttle suture may be coupled with a first needle, and a second end of the shuttle suture may be coupled with a second needle.

According to an aspect of the present disclosure, a shuttling feature may be a shuttle link defined proximate to a second end.

According to an aspect of the present disclosure, a shuttle suture may have a bifurcated section that defines a shuttle link.

According to an aspect of the present disclosure, a shuttling feature may be a shuttle receiving area.

According to an aspect of the present disclosure, a shuttle suture may be constructed of multiple strands. A repair suture is configured to be inserted between the multiple strands at a shuttle receiving area.

According to an aspect of the present disclosure, a first end and a second end of a shuttle suture may be coupled to a first needle.

According to an aspect of the present disclosure, a shuttle suture may be folded to form two limbs that extend through a pledget.

According to an aspect of the present disclosure, a shuttling feature may be a shuttle loop including a mid-section of a shuttle suture.

According to an aspect of the present disclosure, a mid-section of the shuttle suture forming a shuttle loop may be operably coupled with a second needle.

According to an aspect of the present disclosure, a shuttle loop may be operably coupled with a second needle via a connecting suture.

According to an aspect of the present disclosure, a method of replacing a cardiovascular valve may include one or more of: inserting a first needle through tissue and a replacement valve, wherein the first needle is coupled to a distal end of a repair suture and a first end of a shuttle suture; inserting a second needle through the tissue and the replacement valve, wherein the second needle is operably coupled to the shuttle suture; positioning a pledget against an inner surface of the tissue; removing the first needle and the second needle from the shuttle suture; inserting the distal end of the repair suture through a shuttling feature of the shuttle suture; applying a tension to at least the first end of the shuttle suture; drawings the shuttling feature and the distal end of the repair suture through the pledget and a locking feature coupled with the pledget; tensioning the repair suture with application of the tension; and locking the repair suture in a stitch configuration by engaging the locking feature.

According to an aspect of the present disclosure, a method may include removing the first needle from the repair suture.

According to an aspect of the present disclosure, a method may include a step of removing a first needle and a second needle from a shuttle suture, which may include cutting a connecting suture and removing the second needle and the connecting suture from the shuttle suture.

According to an aspect of the present disclosure, a method may include a step of applying a tension to at least a first end of a shuttle suture, which may include applying the tension to both the first end and a second end of the shuttle suture.

According to an aspect of the present disclosure, a method may include a step of inserting a distal end of a repair suture through a shuttling feature, which may include inserting the distal end through a folded shuttle loop formed from folding a shuttle suture.

According to an aspect of the present disclosure, a method may include a step of inserting a second needle through a tissue and a replacement valve, which may include drawing a mid-section of a shuttle suture through the tissue and the replacement valve with a second needle.

According to an aspect of the present disclosure, a method may include a step of inserting a second needle through a tissue and a replacement valve, which may include drawing a second end of a shuttle suture through the tissue and the replacement valve with a second needle.

According to an aspect of the present disclosure, a step of inserting a distal end of a repair suture through a shuttling feature may include inserting the distal end through a bifurcated shuttle link.

According to an aspect of the present disclosure, a shuttle suture may be formed of multiple strands of material. A method may include a step of inserting the distal end of a repair suture through a shuttling feature, which may include inserting the distal end between the multiple strands of material in a shuttle receiving area.

According to an aspect of the present disclosure, a locking feature may be a spliced section defined by a repair suture. A step of locking the repair suture in a stitch configuration may include drawing the repair suture through the spliced section and bunching stuffing in the repair suture proximate to the spliced section.

According to an aspect of the present disclosure, a locking feature may be a grommet with inner barbs. A step of locking a repair suture in a stitch configuration may include drawing the repair suture through the grommet and engaging the inner barbs.

According to an aspect of the present disclosure, a knotless suture assembly may include a repair suture including a first end and a second end and a shuttle suture having a first end and a second end. The first end may form a shuttling loop. The shuttle suture may be spliced into the repair suture at a first spliced segment proximate and a second spliced segment. A stuffing suture may be spliced into the repair suture between the first and second spliced segments. The shuttle suture may be configured to guide the first end of the repair suture through the first spliced segment which can, consequently, bunch the stuffing suture to lock the repair suture in a stitch configuration.

According to an aspect of the present disclosure, a first spliced segment may be proximate to a first end of a repair suture and a first end of a shuttle suture.

According to an aspect of the present disclosure, a second spliced segment may be proximate to a second end of a repair suture and a second end of a shuttle suture.

According to an aspect of the present disclosure, a second end of a shuttle suture may be encapsulated in a repair suture proximate to a second end of the repair suture.

According to an aspect of the present disclosure, a first spliced segment may be disposed proximate to a first transition section between multiple limbs of a repair suture on a first side of a mid-region of the repair suture. A second spliced segment may be disposed proximate to a second transition section between multiple limbs of the repair suture on a second side of the mid-region of the repair suture.

According to an aspect of the present disclosure, a mid-region may have a greater thickness than each of multiple limbs on each of first and second sides thereof.

According to an aspect of the present disclosure, first and second ends of a repair suture are tapered.

According to an aspect of the present disclosure, a method of manufacturing a suture assembly may include one or more of: cutting suture material to a predefined length; forming a repair suture with a mid-region, multiple limbs on a first side of the mid-region, and multiple limbs on a second side of the mid-region; splicing a shuttle suture into the repair suture at a first location; splicing a stuffing suture into the mid-region of the repair suture; and splicing the shuttle suture into the repair suture at a second location spaced from the first location; forming a first end of the repair suture with the multiple limbs on the first side; and forming a second end of the repair suture with the multiple limbs on the second side.

According to an aspect of the present disclosure, a method may include positioning a shuttle loop of the shuttle suture proximate to the first end of the repair suture and positioning a non-looped end of the shuttle suture proximate to the second end of the repair suture.

According to an aspect of the present disclosure, a step of splicing a shuttle suture into a repair suture at a first location may include splicing the shuttle suture into the repair suture proximate to a transition section between a mid-region and multiple limbs on a first side.

According to an aspect of the present disclosure, a step of splicing a shuttle suture into a repair suture at a second location may include splicing the shuttle suture into the repair suture proximate to a transition section between a mid-region and multiple limbs on a second side.

According to an aspect of the present disclosure, first and second locations may be on opposing sides of a mid-region.

According to an aspect of the present disclosure, a step of splicing a stuffing suture into a mid-region may include increasing a thickness of a repair suture at the mid-region.

According to an aspect of the present disclosure, a step of forming a repair suture may include splicing suture segments together to form multiple limbs on opposing sides of a mid-region.

According to an aspect of the present disclosure, a step of forming a repair suture may include bifurcating suture material to form multiple limbs.

According to an aspect of the present disclosure, a method of compressing tissue bodies may include one or more of: providing a suture assembly with a shuttle suture spliced into a repair suture to include a spliced segment; inserting a suture assembly through a first tissue body in a first direction; inserting the suture assembly through a second tissue body in a second opposing direction; engaging an end of the repair suture with a shuttle loop of the shuttle suture; guiding the end of the repair suture through the spliced segment; and applying a tension force to the end of the repair suture; compressing first and second tissue bodies together with the repair suture; and locking the repair suture under tension.

According to an aspect of the present disclosure, a method may include inserting the suture assembly through the second tissue body in the first direction and inserting the suture assembly through the first tissue body in the second opposing direction.

According to an aspect of the present disclosure, a first tissue body overlaps a second tissue body.

According to an aspect of the present disclosure, a first tissue body is laterally adjacent to a second tissue body.

According to an aspect of the present disclosure, a knotless suture assembly may include a repair suture including a mid-region between a first end and a second end and a shuttle suture including a mid-section between a first end and a second end. The first end of the shuttle suture may be spliced into the repair suture at a first spliced segment. The second end of the shuttle suture may include a shuttling feature spliced into the repair suture at a second spliced segment. A locking feature may include a third spliced segment formed between the mid-section and the mid-region. The locking feature may be configured to retain the repair suture under tension in response to a shuttling process. A first needle may be coupled to at least the first end of the repair suture. A second needle may be coupled to at least the second end of the repair suture.

According to an aspect of the present disclosure, a repair suture may extend beyond first and second ends of a shuttle suture to engage first and second needles, respectively.

According to an aspect of the present disclosure, at least one of first and second ends of a repair suture may be tapered to have a lesser thickness than a mid-region.

According to an aspect of the present disclosure, a pledget may be operably coupled with a locking feature.

According to an aspect of the present disclosure, a stuffing suture may be spliced into a repair suture at a spliced region proximate to a locking feature to increase a thickness of the repair suture at the spliced region.

According to an aspect of the present disclosure, a spliced region may be disposed between a locking feature and a first spliced segment.

According to an aspect of the present disclosure, first and second ends of a shuttle suture may be encapsulated in a repair suture. First and second ends of the shuttle suture may be configured to be separated from a repair suture in response to removal of first and second According to an aspect of the present disclosure, a shuttling feature may be an end shuttle loop.

According to an aspect of the present disclosure, a method of manufacturing a knotless suture assembly may include one or more of: forming a repair suture with a mid-region between a first end and a second end; providing a shuttle suture with a first end and a second end with a shuttling feature; splicing the first end of the shuttle suture into the repair suture at a first location proximate to the first end of the repair suture; forming a first spliced segment at the first location between the shuttle suture and the repair suture; splicing the shuttle suture into the repair suture at a second location spaced from the first location; forming a second spliced segment between the shuttle suture and the repair suture at the second location; forming a locking feature with the second spliced segment for retaining the repair suture under tension in response to a shuttling process; and coupling the first end of the repair suture with a first needle.

According to an aspect of the present disclosure, a method may include splicing a second end of a shuttle suture into a repair suture proximate to a second end of the repair suture and forming a third spliced segment between the shuttle suture and the repair suture at a third location.

According to an aspect of the present disclosure, a method may include coupling a second end of a repair suture with a second needle.

According to an aspect of the present disclosure, a method may include splicing a stuffing suture into a mid-region of a repair suture on an opposing side of a locking feature relative to a shuttling feature.

According to an aspect of the present disclosure, a method may include forming a first end of the repair suture with multiple limbs on a first side of a mid-region and forming a second end of the repair suture with multiple limbs on a second side of the mid-region.

According to an aspect of the present disclosure, a step of splicing a shuttle suture into a repair suture at the second location may include splicing a mid-section of the shuttle suture into a mid-region of the repair suture.

According to an aspect of the present disclosure, a method may include removing a first needle and separating a first end of a shuttle suture from a first end of a repair suture.

According to an aspect of the present disclosure, a knotless suture assembly may include a repair suture including a first end and a second end and a shuttle suture having a first end and a second end. The second end of the shuttle suture may form a shuttling feature. The shuttle suture may be spliced into the repair suture at a first spliced segment proximate to the first end of the repair suture and at a second spliced segment. The second spliced segment forms at least a portion of a locking feature. A stuffing suture may be spliced into the repair suture between the first and second spliced segments. The shuttling feature may be configured to guide the first end of the repair suture through the locking feature in response to a pulling force being applied to the first end of the shuttle suture which may, consequently, bunch the stuffing suture to lock the repair suture in a stitch configuration. At least one needle may be coupled with the repair suture.

According to an aspect of the present disclosure, a second end of a shuttle suture with a shuttling feature may be spliced into a repair suture proximate to a second end of the repair suture.

According to an aspect of the present disclosure, at least one needle may include a first needle coupled to a first end of a repair suture and a second needle coupled to a second end of the repair suture.

According to an aspect of the present disclosure, a pledget may be operably coupled with a repair suture proximate to a locking feature.

According to an aspect of the present disclosure, first and second ends of a repair suture may be tapered.

According to an aspect of the present disclosure, a method of attaching a cardiovascular replacement valve to tissue may include one or more of: inserting a first needle through tissue and a replacement valve; carrying a first end of a repair suture and a first end of a shuttle suture through the tissue and the replacement valve with the first needle; inserting a second needle through the tissue and the replacement valve; carrying a second end of the shuttle suture through the tissue and the replacement valve with the second needle; positioning a pledget against an inner surface of the tissue; removing the first and second needles; inserting the first end of the repair suture through a shuttling feature at the second end of the shuttle suture; applying a pulling force to the first end of the shuttle suture; pulling the shuttling feature and the first end of the repair suture through a locking feature; and locking the repair suture under tension with the locking feature.

According to an aspect of the present disclosure, a method may include providing a shuttle suture having a shuttling feature configured as a bifurcated segment forming a shuttle link.

According to an aspect of the present disclosure, a step of inserting a first end of a repair suture through a shuttling feature may include inserting the first end of the repair suture through a shuttle link.

According to an aspect of the present disclosure, a step of inserting a first end of a repair suture through a shuttling feature may include inserting the first end of the repair suture between strands of a shuttle suture at a shuttle receiving area.

According to an aspect of the present disclosure, a method may include one or more of: pulling a first end of a repair suture across a replacement valve, through the replacement valve, through tissue and to a locking feature; pulling the first end of the repair suture from the locking feature, through the tissue, and through the replacement valve; forming a stitch configuration with the repair suture; and sealing the replacement valve to the tissue with the repair suture in the stitch configuration.

According to an aspect of the present disclosure, a method may include tensioning a repair suture and re-tensioning the repair suture, wherein a locking feature may be configured to lock the repair suture under tension after each re-tensioning of the repair suture.

According to an aspect of the present disclosure, a method may include carrying a second end of a repair suture through tissue and a replacement valve with a second needle.

According to an aspect of the present disclosure, a locking feature may be a spliced section defined by a repair suture. A step of locking the repair suture under tension may include one or more of: drawing the repair suture through the spliced section; forming a stitch configuration with the repair suture; and bunching stuffing in the repair suture proximate the spliced section.

According to an aspect of the present disclosure, a locking feature may be a grommet with inner barbs. A step of locking the repair suture in under tension may include one or more of: drawing a repair suture through the grommet; forming a stitch configuration with the repair suture; and engaging the inner barbs.

According to an aspect of the present disclosure, a method of replacing a cardiovascular valve may include one or more of: inserting a first needle through tissue and a replacement valve; carrying an end of a repair suture, a first end of a shuttle suture, and a second end of the shuttle suture through the tissue and the replacement valve with the first needle; inserting a second needle through the tissue and the replacement valve; carrying a mid-section of the shuttle suture through the tissue and the replacement valve with the second needle; positioning a pledget against an inner surface of the tissue; removing the first needle and the second needle; inserting the end of the repair suture through a shuttling feature of the shuttle suture; applying a tension to at least the first end of the shuttle suture; drawing the shuttling feature and the end of the repair suture through a locking feature operably coupled with the pledget; tensioning the repair suture; and locking the repair suture under tension in a stitch configuration in response to an engagement between the repair suture and the locking feature.

According to an aspect of the present disclosure, a step of removing a first needle and a second needle may include one or more of: removing the first needle from a shuttle suture and a repair suture; cutting a connecting suture; and removing the second needle and the connecting suture from the shuttle suture.

According to an aspect of the present disclosure, a step of applying tension to at least a first end of a shuttle suture may include applying the tension to both the first end and a second end of the shuttle suture and pulling the mid-section of the shuttle suture through a locking feature.

According to an aspect of the present disclosure, a step of inserting an end of a repair suture through a shuttling feature may include inserting the end through a folded shuttle loop formed from folding a shuttle suture at a mid-section.

According to an aspect of the present disclosure, a locking feature may be a spliced section defined by a repair suture. A step of locking the repair suture in a stitch configuration may include drawing the repair suture through the spliced section and bunching stuffing in the repair suture proximate to the spliced section.

According to an aspect of the present disclosure, a locking feature may be a grommet with inner barbs. A step of locking the repair suture in a stitch configuration may include drawing the repair suture through the grommet and engaging the inner barbs.

According to an aspect of the present disclosure, a method of compressing cardiovascular tissue bodies may include one or more of: providing a suture assembly with a repair suture and a shuttle suture; inserting an end of the suture assembly through a first tissue body in a first direction; inserting the end of the suture assembly through a second tissue body in a second opposing direction; engaging an end of the repair suture with a shuttling feature of the shuttle suture; a spliced segment between the shuttle suture and the repair suture; applying a tension force to the end of the repair suture; compressing first and second tissue bodies together with the repair suture; and locking the repair suture under tension.

According to an aspect of the present disclosure, a method may include inserting a suture assembly through a second tissue body in a first direction and inserting the suture assembly through a first tissue body in a second opposing direction.

According to an aspect of the present disclosure, a step of inserting an end a suture assembly through a second tissue body may include inserting the end of the suture assembly through the second tissue body that overlaps a first tissue body.

According to an aspect of the present disclosure, a step of inserting an end a suture assembly through a second tissue body may include inserting the end of the suture assembly through the second tissue body that is laterally adjacent to a first tissue body.

According to an aspect of the present disclosure, a first tissue body overlaps a second tissue body.

According to an aspect of the present disclosure, a first tissue body is laterally adjacent to a second tissue body.

According to an aspect of the present disclosure, a method may include providing a suture assembly with a shuttle suture spliced into a repair suture to include at least one spliced segment.

According to an aspect of the present disclosure, a step of providing a suture assembly may include providing the suture assembly with a shuttle suture spliced into a repair suture at multiple locations.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

The claims:

1. A knotless suture assembly, comprising:
a repair suture including a mid-region between a first end and a second end;
a shuttle suture including a mid-section between a first end and a second end, wherein the first end of the shuttle suture is spliced into the repair suture at a first spliced segment, and wherein the second end of the shuttle suture includes a shuttling feature spliced into the repair suture at a second spliced segment;
a locking feature including a third spliced segment formed between the mid-section and the mid-region, the locking feature configured to retain the repair suture under tension in response to a shuttling process;
a first needle coupled to at least the first end of the repair suture; and
a second needle coupled to at least the second end of the repair suture.

2. The knotless suture assembly of claim 1, wherein the repair suture extends beyond the first and second ends of the shuttle suture to engage the first and second needles, respectively.

3. The knotless suture assembly of claim 1, wherein at least one of the first and second ends of the repair suture are tapered to have a lesser thickness than the mid-region.

4. The knotless suture assembly of claim 1, further comprising:
a pledget operably coupled with the locking feature.

5. The knotless suture assembly of claim 1, further comprising:
a stuffing suture spliced into the repair suture at a spliced region proximate to the locking feature to increase a thickness of the repair suture at the spliced region.

6. The knotless suture assembly of claim 5, wherein the spliced region is disposed between the locking feature and the first spliced segment.

7. The knotless suture assembly of claim 1, wherein the first and second ends of the shuttle suture are encapsulated in the repair suture, and wherein the first and second ends of the shuttle suture are configured to be separated from the repair suture in response to removal of the first and second needles.

8. The knotless suture assembly of claim 1, wherein the shuttling feature is an end shuttle loop.

9. A method of manufacturing a knotless suture assembly, comprising:
forming a repair suture with a mid-region between a first end and a second end;
providing a shuttle suture with a first end and a second end with a shuttling feature;
splicing the first end of the shuttle suture into the repair suture at a first location proximate to the first end of the repair suture;
forming a first spliced segment at the first location between the shuttle suture and the repair suture;
splicing the shuttle suture into the repair suture at a second location spaced from the first location;
forming a second spliced segment between the shuttle suture and the repair suture at the second location;
forming a locking feature with the second spliced segment for retaining the repair suture under tension in response to a shuttling process; and
coupling the first end of the repair suture with a first needle.

10. The method of claim 9, further comprising:
splicing the second end of the shuttle suture into the repair suture proximate to the second end of the repair suture; and
forming a third spliced segment between the shuttle suture and the repair suture at a third location.

11. The method of claim 10, further comprising:
coupling the second end of the repair suture with a second needle.

12. The method of claim 9, further comprising:
splicing a stuffing suture into the mid-region of the repair suture on an opposing side of the locking feature relative to the shuttling feature.

13. The method of claim 9, further comprising:
forming the first end of the repair suture with multiple limbs on a first side of the mid-region; and
forming the second end of the repair suture with multiple limbs on a second side of the mid-region.

14. The method of claim 9, wherein the step of splicing the shuttle suture into the repair suture at the second location includes splicing a mid-section of the shuttle suture into the mid-region of the repair suture.

15. The method of claim 9, further comprising:
removing the first needle; and
separating the first end of the shuttle suture from the first end of the repair suture.

16. A knotless suture assembly, comprising:
a repair suture including a first end and a second end;
a shuttle suture having a first end and a second end, the second end of the shuttle suture forming a shuttling feature, wherein the shuttle suture is spliced into the repair suture at a first spliced segment proximate the first end of the repair suture and at a second spliced segment, wherein the second spliced segment forms at least a portion of a locking feature;
a stuffing suture spliced into the repair suture between the first and second spliced segments, wherein the shuttling feature is configured to guide the first end of the repair suture through the locking feature in response to a pulling force being applied to the first end of the shuttle suture which, consequently, bunches the stuffing suture to lock the repair suture in a stitch configuration; and
at least one needle coupled with the repair suture.

17. The knotless suture assembly of claim 16, wherein the second end of the shuttle suture with the shuttling feature is spliced into the repair suture proximate to the second end of the repair suture.

18. The knotless suture assembly of claim 16, wherein the at least one needle includes a first needle coupled to the first end of the repair suture and a second needle coupled to the second end of the repair suture.

19. The knotless suture assembly of claim 16, further comprising:
a pledget operably coupled with the repair suture proximate to the locking feature.

20. The knotless suture assembly of claim 16, wherein the first and second ends of the repair suture are tapered.
